SEQUENCE LISTING (6 sequences)

SEQ ID NO: 1 - DNA, 26 bp, Artificial
  Primer
tgtcttcctcgtcctgctgttcctcg

SEQ ID NO: 2 - DNA, 27 bp, Artificial
  Primer
ctgcctcgtatatgaaaccaccatcaa

SEQ ID NO: 3 - DNA, 30 bp, Artificial
  Primer
cgacagtcagccgcatcttcttttgcgtcg

SEQ ID NO: 4 - DNA, 30 bp, Artificial
  Primer
ggactgtggtcatgagtccttccacgatac

SEQ ID NO: 5 - DNA, 2390 bp, Homo sapiens
agagccttcgtttgccaagtcgcctccagaccgcagacatgaaacttgtcttcctcgtcctgctgttcctcggggccctcggactgtgtctggctggccgtaggaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggcctcccctgctgaaggtggggattgcccatccatctgcttacaattccctgctgtcgtcttagcaagaagtaaaatgagaaattttgttgatattctctccttaaaaaaaaaaaaaaaaaaaaaaaaaaaaaa

SEQ ID NO: 6 - protein, 710 aa, Homo sapiens
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

(19) United States
(12) Patent Application Publication
(10) Pub. No.: US 2011/0182914 A1
(43) Pub. Date: Jul. 28, 2011

(54) METHODS AND COMPOSITIONS

(76) Inventors: Christopher Gregory, Edinburgh (GB); John David Pound, Edinburgh (GB); Irini Bournazou, Edinburgh (GB)

(21) Appl. No.: 13/000,089

(22) PCT Filed: Jun. 18, 2009
(86) PCT No.: PCT/GB09/01550

§ 371 Date: Mar. 28, 2011

(30) Foreign Application Priority Data
Jun. 19, 2008 (GB) 0811250.0

(51) Int. Cl. A61K 39/395; C12N 5/0787; A61K 38/40; C07H 21/00; C07K 16/18; A61K 31/7088; A61P 29/00; A61P 35/00; A61P 11/00

(52) U.S. Cl. 424/172.1; 435/375; 514/1.1; 536/23.5; 530/389.1; 536/24.5; 514/44 A; 514/44 R

(57) ABSTRACT

The invention relates to methods for modulating granulocyte activation and migration, use of such methods in the treatment of diseases and compositions which may be employed in such methods and uses. In particular, the modulation of granulocyte activation/migration is achieved by increasing or decreasing the amount of lactoferrin in the vicinity of said granulocytes.

### FIELD OF THE INVENTION

The present invention relates to methods for modulating granulocyte, for example neutrophil, activation and migration, use of such methods in the treatment of inflammatory diseases and the treatment of cancer, and compositions which may be employed in such methods and uses.

### BACKGROUND OF THE INVENTION

Granulocytes are a class of leukocytes characterized by prominent cytoplasmic granules. There are three major granulocyte cell types: neutrophils, eosinophils and basophils.

Neutrophils, the most abundant leukocytes in the circulation, provide a first line of defence against invading pathogens through phagocytosis together with activation and release of antimicrobial compounds. Although positive attraction molecules for neutrophils have been well-characterised (eg. leukotrienes, cytokines such as IL-8 and bacterial components such as fMLP), little is known to date with respect to negative modulators of neutrophil migration. Negative modulators identified to date are lipoxins, netrins (netrin-1), annexin-1, resolvins and protectins. Of these, many are not neutrophil specific; Netrin-1 is also involved in inhibition of monocytes, lymphocytes and neuronal cell migration; annexin-1 is also involved in epithelial cell migration.

Neutrophils are rapidly recruited to inflammatory sites in response to positive chemotactic signals, such as host chemokines and microbial chemoattractants. However, while it is accepted that neutrophil infiltration is subject to tight regulation, possible mechanisms that counterbalance the positive chemoattractive processes, thereby preventing excessive neutrophil infiltration through negative feedback, have received little attention. Furthermore, amongst ‘professional’ phagocytes, neutrophils, in stark contrast to macrophages, are not normally recruited to sites of apoptosis where dying host cells are cleared through phagocytosis.

### SUMMARY OF THE INVENTION

The present inventors have examined regulation of granulocyte activation and have surprisingly demonstrated that lactoferrin acts as a potent inhibitor of granulocyte migration.

The demonstration that lactoferrin inhibits granulocyte activation enables the use of lactoferrin modulators, either enhancers or inhibitors of lactoferrin expression or activity, in the modulation of granulocyte activity. Accordingly, in a first aspect of the present invention, there is provided a method of modulating granulocyte activation and/or migration towards a cell or population of cells, said method comprising modulating the amount or activity of lactoferrin in the vicinity of said cells and/or said granulocytes.

As described in the Examples, the inventors have demonstrated that, upon induction of apoptosis, cells synthesise and release lactoferrin that acts as a potent inhibitor of neutrophil migration both in vitro and in vivo. In addition, the inventors have shown that lactoferrin that acts as a potent inhibitor of migration of other granulocytes, such as eosinophils.

Thus, in one embodiment of the invention, the modulation of granulocyte migration is inhibition of granulocyte migration towards said cell or population of cells. In one embodiment, the inhibition is achieved by increasing the amount of lactoferrin in the vicinity of said cells and/or granulocytes.

In one embodiment of the invention the granulocytes are neutrophils. In another embodiment, the granulocytes are eosinophils.

As shown in the Examples, induction of apoptosis in a panel of cell types of divergent lineages results in substantial upregulation of lactoferrin expression at both transcriptional and protein levels. In the invention, an increase in the amount of lactoferrin may be achieved by any suitable means known to the skilled person. For example, the amount or concentration of lactoferrin may be increased by administration of lactoferrin or nucleic acid encoding lactoferrin to said cells.

Upon activation, CD62L is cleaved from neutrophil surface whereas CD11b expression is upregulated following translocation from cytoplasmic granules to the cell membrane. As shown in the Examples, each of these effects is inhibited by lactoferrin.

In one embodiment of the invention, inhibition of granulocyte migration is accompanied by reduced polarisation of granulocytes and/or a reduction of cleavage of CD62L and reduction of expression of CD11b when compared to granulocytes in the absence of enhanced amounts of lactoferrin.

Indeed, in a second independent aspect of the invention, there is provided a method of inhibiting polarisation of granulocytes, said method comprising administration of lactoferrin or nucleic acid encoding lactoferrin to said granulocytes.

Furthermore, the inventors have also shown that the effect of lactoferrin on chemotaxis towards a range of chemoattractants was granulocyte specific with no significant effect demonstrated on monocyte and macrophage migration. Accordingly, in one embodiment of the invention, the modulation of lactoferrin does not modulate the migration of macrophages or monocytes.

The inventors' results therefore reveal for the first time, a novel immunoregulatory function for lactoferrin and identify it as one of only a few molecules to negatively regulate leukocyte migration. Moreover, the effect of lactoferrin appears to be granulocyte specific.

Without being limited to any one theory, the inventors believe that the results suggest that lactoferrin production and release at sites of apoptosis contributes to the non-phlogistic nature of the apoptosis program by mediating exclusion of granulocytes.

The demonstration that lactoferrin has a modulatory effect on granulocyte, for example neutrophil, migration and, in particular, that its production by apoptotic cells regulates granulocyte, for example neutrophil, infiltration to sites of apoptosis identifies lactoferrin as a mediator of resolution of inflammation and as a therapeutic target with potential to control granulocyte infiltration in inflammatory and malignant diseases. The demonstration of the granulocyte specificity of lactoferrin is of particular advantage, as it suggests that lactoferrin can be manipulated as a therapeutic target specific for granulocytes in inflammatory conditions characterised by aberrant granulocyte infiltration without affecting the whole immune cell response or trigger an immunosuppressive situation. This cell specificity also indicates that this natural modulator is non-toxic to the host.

Accordingly in a third aspect of the present invention, there is provided a method of treating inflammatory disease, said method comprising administering a modulator of lactoferrin concentration to a subject in need thereof. In this aspect of the invention, the modulator of lactoferrin concentration preferably increases lactoferrin concentration in a target site, e.g. the site of inflammation.

The invention is of particular use in the treatment of inflammatory diseases, such as chronic inflammatory diseases, associated with excessive granulocyte infiltration and granulocyte-mediated tissue damage and remodelling. Thus, in one embodiment of the third aspect of the invention, the inflammatory disease is a chronic inflammatory disease. Examples of such chronic inflammatory disease include, but are not limited to vasculitis, pulmonary fibrosis, and ischaemia reperfusion injury.

The invention may also be used in the treatment of various tumours. Accordingly, in a fourth aspect of the invention, there is provided a method of treating cancer in a subject, said method comprising administering a lactoferrin modulator to a target site in said subject.

In certain tumours, neutrophils may play a supportive role. Evidence for their tumour-enhancing role is supported by the strong correlation between tumour grade and extent of neutrophil infiltration. For example, gliomas, a primary central nervous system tumour that arises from glial cells, verrucous and gastric carcinomas as well as many primary and metastatic melanomas are all previously described by a massive neutrophil infiltration. In all these cases, a tumour microenvironment is formed that recruits neutrophils, while in parallel, angiogenesis is enhanced and tumour growth and invasion are promoted via the production of pro-angiogenic factors e.g. VEGF and IL-8, elastases and proteases such as matrix metalloproteinases. Thus, in tumours in which neutrophils or other granulocytes play a supportive role, the administration of agents which increase lactoferrin in the vicinity of the tumour and thus inhibit neutrophil (or other granulocyte) migration to said tumour cells may be of considerable therapeutic benefit. Thus, in one embodiment of the fourth aspect of the invention, the lactoferrin modulator enhances the concentration, expression or activity of lactoferrin at the target site. In one such embodiment, the cancer is a selected from the group comprising gliomas, verroucous carcinoma, gastric carcinoma and melanoma. In the majority of tumours, however, neutrophils are absent and are not believed to provide such a supportive role. Thus, given the well-known oncolytic effects of neutrophils, encouragement of neutrophil infiltration through inhibition of lactoferrin may be used to effect tumour destruction. Accordingly, in one embodiment of the fourth aspect of the invention, the modulator of lactoferrin concentration reduces the concentration, expression or activity of lactoferrin at a target site. Thus, in such embodiments, the lactoferrin modulator is a lactoferrin inhibitor.

A fifth aspect of the present invention provides a lactoferrin inhibitor for use in medicine.

A sixth aspect of the invention provides a modulator of lactoferrin concentration or expression for use in a method of treating an inflammatory disease, for example chronic inflammatory disease. Also encompassed by the sixth aspect of the present invention is the use of a lactoferrin modulator in the preparation of a medicament for the treatment of inflammatory disease.

A seventh aspect of the invention provides a modulator of lactoferrin concentration or expression of lactoferrin for use in the treatment of cancer. Also encompassed by the seventh aspect of the present invention is the use of a modulator of lactoferrin concentration or expression in the preparation of a medicament for the treatment of cancer. In embodiments of this aspect of the invention, wherein the modulator is an enhancer of lactoferrin activity or concentration, the cancer is a cancer in which neutrophils play a supportive role, for example a cancer is selected from the group comprising, but not limited to, gliomas, verroucous carcinoma, gastric carcinoma and melanoma.

According to an eighth aspect of the invention, there is provided a pharmaceutical composition comprising a modulator of lactoferrin concentration or expression.

The pharmaceutical composition of the eighth aspect of the invention may be used in the treatment of any condition for which modulation of granulocyte migration may be beneficial. In one embodiment, the pharmaceutical composition is for treatment of inflammatory disease. In another embodiment, the pharmaceutical composition is for use in the treatment of cancer.

It should be understood that references to modulators of lactoferrin concentration and/or expression as used herein, may take the form of small organic molecules, proteins, peptides (including fragments, portions, analogues or derivatives of lactoferrin), amino acids, nucleic acids (RNA or DNA: including sense or antisense sequences) and/or antibodies (or antigen binding fragments thereof). In the case of inhibitors of lactoferrin concentration and/or expression, antibodies or antigen binding fragments thereof (for example Fab, F(ab)₂, or nanobodies etc) which exhibit a specificity/affinity for, or selectivity to, lactoferrin or one or more epitope(s) thereof, may be particularly useful. One of skill will readily understand that antibodies may be polyclonal antibodies generated by immunisation with specific antigens, or monoclonal antibodies. The techniques and/or procedures used to generate antibodies are further described in “Antibodies: A Laboratory Manual: 1988 Cold Spring Harbor Lab.)

In a further embodiment, compounds capable of inhibiting lactoferrin concentration and/or expression may include, for example, DNA or RNA oligonucleotides, preferably antisense oligonucleotides. In one embodiment, the oligonucleotides may be RNA molecules known to those skilled in this field as small/short interfering and/or silencing RNA and which will be referred to hereinafter as siRNA. Such siRNA oligonucleotides may take the form of native RNA duplexes or duplexes which have been modified in some way (for example by chemical modification) to be nuclease resistant. Additionally, or alternatively, the siRNA oligonucleotides may take the form of short hairpin RNA (shRNA) expression or plasmid constructs.

The skilled man will readily understand that antisense oligonucleotides may be used to modulate (for example, inhibit, down-regulate or substantially ablate) the expression of any given gene. Accordingly, (antisense) oligonucleotides provided by this invention may be designed to modulate, i.e. inhibit or neutralise, the expression and/or function of the lactoferrin gene and/or its protein product.

By analysing native or wild-type lactoferrin sequences and with the aid of algorithms such as BIOPREDsi, one of skill in the art could easily determine or computationally predict nucleic acid sequences that have an optimal knockdown effect for these genes (see for example: http://www.biopredsi.org/start.html). Accordingly, the skilled man may generate and test an array or library of different oligonucleotides to determine whether or not they are capable of modulating the expression or function of lactoferrin genes and/or proteins.

Preferred and alternative features of each aspect of the invention are as for each of the other aspects mutatis mutandis unless the context demands otherwise.

### DETAILED DESCRIPTION

As described above and in the Examples, the present invention is based on the demonstration that apoptotic cells express lactoferrin and that lactoferrin inhibits granulocyte migration towards such cells. The demonstration of the modulatory effect of lactoferrin on granulocytes enables the use of lactoferrin modulation in the manipulation of granulocyte behaviour and in the use of lactoferrin and lactoferrin modulators in a number of therapeutic contexts.

In the context of the present application, a lactoferrin modulator may be a lactoferrin enhancer or a lactoferrin inhibitor. In the context of the present invention, a lactoferrin enhancer is a modulator which increases lactoferrin concentration, expression or activity. Preferably, such lactoferrin enhancers specifically increase lactoferrin concentration, expression or activity. Examples of suitable modulators include but are not limited to lactoferrin, lactoferrin analogues, nucleic acid encoding lactoferrin or lactoferrin analogues, or enhancers of lactoferrin expression or activity. In a particular embodiment, the lactoferrin enhancer is lactoferrin or a nucleic acid encoding lactoferrin.

Lactoferrin analogues for use in the invention means a polypeptide modified by varying the amino acid sequence of a wild-type lactoferrin molecule e.g. by manipulation of the nucleic acid encoding the protein or by altering the protein itself, wherein said analogue has lactoferrin biological activity i.e. granulocyte, for example neutrophil, migration inhibitory activity and/or ability to promote proliferation.

Such analogues may involve substitution or deletion, for example of 50 or fewer amino acids, more preferably of 40 or fewer, even more preferably of 25 or fewer, most preferably of 1 to 5 amino acids only and/or the insertion or addition of 50 or fewer amino acids, more preferably of 40 or fewer, even more preferably of 25 or fewer, most preferably of 1 to 5 amino acids or less amino acid residues. In another embodiment, a lactoferrin analogue may share at least 70%, for example at least 80%, such as at least 90%, at least 95% or at least 99% sequence homology with the full-length wild-type lactoferrin. The amino acid sequence for lactoferrin is shown in FIG. 10(b). In one embodiment, the lactoferrin analogue may be delta lactoferrin, a truncated form produced from alternative splicing (Siebert and Huang, PNAS, 94, 2198-2203).

Analogues of the invention also include multimeric peptides including such peptides and prodrugs including such sequences, derivatives of the peptides of the invention, including the peptide linked to a coupling partner, e.g. an effector molecule, a label, a drug, a toxin and/or a carrier or transport molecule. Techniques for coupling lactoferrin peptides to both peptidyl and non-peptidyl coupling partners are well known in the art.

Analogues of the invention include fusion peptides. For example, analogues may comprise peptides of the invention linked, for example, to antibodies that target the peptides to diseased tissue, for example, heart tissue or tumour tissue.

The peptides described herein may be fused with the constant domain of immunoglobulins (IgA, IgE, IgG, IgM), or portions thereof (CH1, CH2, CH3, or any combination thereof) and portions thereof, resulting in chimeric polypeptides. These fusion proteins can facilitate purification and show an increased half-life in vivo. Such fusion proteins may be more efficient in binding and neutralizing other molecules than monomeric polypeptides or fragments thereof alone. See, e.g., Fountoulakis et al., J. Biochem., 270:3958-3964 (1995).

Fusion proteins for use in the invention also include lactoferrin peptides (or analogues) fused with albumin, for example recombinant human serum albumin or fragments or variants thereof (see, e.g., U.S. Pat. No. 5,876,969, EP Patent 0 413 622, and U.S. Pat. No. 5,766,883).

The use of polynucleotides encoding such fusion proteins described herein are also encompassed by the invention.

Analogues for use in the present invention further include reverse-or retro-analogues of natural lactoferrin peptides or their synthetic derivatives. For details relating to reverse peptides, see, for example, EP 0497 366, U.S. Pat. No. 5,519,115, and Merrifield et al., 1995, PNAS, 92:3449-53 for details relating to reverse peptides, the disclosures of which are herein incorporated by reference. As described in EP 0497 366, reverse peptides are produced by reversing the amino acid sequence of a naturally occurring or synthetic peptide. Such reverse-peptides retain the same general three-dimensional structure (e.g., alpha-helix) as the parent peptide except for the conformation around internal protease-sensitive sites and the characteristics of the N-and C-termini. Reverse peptides are purported not only to retain the biological activity of the non-reversed “normal” peptide but may possess enhanced properties, including increased biological activity. (See Iwahori et al., 1997, Biol. Pharm. Bull. 20: 267-70). Analogues of and for use in the present invention may therefore comprise reverse peptides of natural and synthetic QUB 919 peptides.

In some embodiments of the invention, the lactoferrin modulators are lactoferrin inhibitors. Any suitable inhibitor may be used. In the present invention, any molecule which reduces expression of a lactoferrin gene or antagonizes a lactoferrin peptide may be used as the lactoferrin inhibitor. Such inhibitors may include, but are not limited to, antibodies, antibody fragments, immunoconjugates, small molecule inhibitors, peptide inhibitors, specific binding members, non-peptide small organic molecules, nucleic acid modulators such as antisense molecules siRNA molecules or oligonucleotide decoys.

Antibodies

Antibodies and antibody fragments for use in the present invention may be produced in any suitable way, either naturally or synthetically. Such methods may include, for example, traditional hybridoma techniques (Kohler and Milstein (1975) Nature, 256:495-499), recombinant DNA techniques (see e.g. U.S. Pat. No. 4,816,567), or phage display techniques using antibody libraries (see e.g. Clackson et al. (1991) Nature, 352: 624-628 and Marks et al. (1992) Bio/Technology, 10: 779-783). Other antibody production techniques are described in Using Antibodies: A Laboratory Manual, eds. Harlow and Lane, Cold Spring Harbor Laboratory, 1999.

Traditional hybridoma techniques typically involve the immunisation of a mouse or other animal with an antigen in order to elicit production of lymphocytes capable of binding the antigen. The lymphocytes are isolated and fused with a myeloma cell line to form hybridoma cells which are then cultured in conditions which inhibit the growth of the parental myeloma cells but allow growth of the antibody producing cells. The hybridoma may be subject to genetic mutation, which may or may not alter the binding specificity of antibodies produced. Synthetic antibodies can be made using techniques known in the art (see, for example, Knappik et al, J. Mol. Biol. (2000) 296, 57-86 and Krebs et al, J. Immunol. Meth. (2001) 2154 67-84.

Modifications may be made in the VH, VL or CDRs of the binding members, or indeed in the FRs using any suitable technique known in the art. For example, variable VH and/or VL domains may be produced by introducing a CDR, e.g. CDR3 into a VH or VL domain lacking such a CDR. Marks et al. (1992) Bio/Technology, 10: 779-783 describe a shuffling technique in which a repertoire of VH variable domains lacking CDR3 is generated and is then combined with a CDR3 of a particular antibody to produce novel VH regions. Using analogous techniques, novel VH and VL domains comprising CDR derived sequences of the present invention may be produced.

Accordingly, antibodies and antibody fragments for use in the invention may be produced by a method comprising: (a) providing a starting repertoire of nucleic acids encoding a variable domain, wherein the variable domain includes a CDR1, CDR2 or CDR3 to be replaced or the nucleic acid lacks an encoding region for such a CDR; (b) combining the repertoire with a donor nucleic acid encoding an amino acid sequence such that the donor nucleic acid is inserted into the CDR region in the repertoire so as to provide a product repertoire of nucleic acids encoding a variable domain; (c) expressing the nucleic acids of the product repertoire; (d) selecting a specific antigen-binding fragment specific for said target; and (e) recovering the specific antigen-binding fragment or nucleic acid encoding it. The method may include an optional step of testing the specific binding member for ability to inhibit the activity of said target.

Alternative techniques of producing antibodies for use in the invention may involve random mutagenesis of gene(s) encoding the VH or VL domain using, for example, error prone PCR (see Gram et al, 1992, P.N.A.S. 89 3576-3580. Additionally or alternatively, CDRs may be targeted for mutagenesis e.g. using the molecular evolution approaches described by Barbas et al 1991 PNAS 3809-3813 and Scier 1996 J Mol Biol 263 551-567.

This therefore enables the use of antibodies and antibody fragments as active therapeutic agents. An antibody for use in the invention may be a “naked” antibody (or fragment thereof) i.e. an antibody (or fragment thereof) which is not conjugated with an “active therapeutic agent”. An “active therapeutic agent” is a molecule or atom which is conjugated to an antibody moiety (including antibody fragments, CDRs etc) to produce a conjugate. Examples of such “active therapeutic agents” include drugs, toxins, radioisotopes, immunomodulators, chelators, boron compounds, dyes etc.

Antibodies for use in the invention herein include antibody fragments and “chimeric” antibodies in which a portion of the heavy and/or light chain is identical with or homologous to corresponding sequences in antibodies derived from a particular species or belonging to a particular antibody class or subclass, while the remainder of the chain (s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81: 6851-6855 (1984)). Chimeric antibodies of interest herein include “primatized”antibodies comprising variable domain antigen-binding sequences derived from a non-human primate (e.g. Old World

Monkey, Ape etc), and human constant region sequences.

A lactoferrin inhibitor for use in the invention may be in the form of an immunoconjugate, comprising an antibody fragment conjugated to an “active therapeutic agent”. The therapeutic agent may be a chemotherapeutic agent or another molecule.

Methods of producing immunoconjugates are well known in the art; for example, see U.S. Pat. No. 5,057,313, Shih et al., Int. J. Cancer 41: 832-839 (1988); Shih et al., Int. J. Cancer 46: 1101-1106 (1990), Wong, Chemistry Of Protein Conjugation And Cross-Linking (CRC Press 1991); Upeslacis et al., “Modification of Antibodies by Chemical Methods, “in Monoclonal Antibodies: Principles And Applications, Birch et al. (eds.), pages 187-230 (Wiley-Liss, Inc. 1995); Price, “Production and Characterization of Synthetic Peptide-Derived Antibodies,” in Monoclonal Antibodies: Production, Engineering And Clinical Application, Ritter et al. (eds.), pages 60-84 (Cambridge University Press 1995).

The antibodies or fragments thereof for use in the invention may comprise further modifications. For example the antibodies can be glycosylated, pegylated, or linked to albumin or a nonproteinaceous polymer.

Nucleic Acid Modulators

Lactoferrin modulators for use in the present invention may comprise nucleic acid molecules capable of modulating gene expression, for example capable of down regulating expression of a sequence encoding a lactoferrin protein. Such nucleic acid molecules may include, but are not limited to antisense molecules, short interfering nucleic acid (siNA), for example short interfering RNA (siRNA), double-stranded RNA (dsRNA), micro RNA, short hairpin RNA (shRNA), nucleic acid sensor molecules, allozymes, enzymatic nucleic acid molecules, and triplex oligonucleotides and any other nucleic acid molecule which can be used in mediating RNA interference “RNAi” or gene silencing in a sequence-specific manner (see for example Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914; Allshire, 2002, Science, 297, 1818-1819; Volpe et al., 2002, Science, 297, 1833-1837; Jenuwein, 2002, Science, 297, 2215-2218; Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831).

An “antisense nucleic acid”, is a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). The antisense molecule may be complementary to a target sequence along a single contiguous sequence of the antisense molecule or may be in certain embodiments, bind to a substrate such that the substrate, the antisense molecule or both can bind such that the antisense molecule forms a loop such that the antisense molecule can be complementary to two or more non-contiguous substrate sequences or two or more non-contiguous sequence portions of an antisense molecule can be complementary to a target sequence, or both. Details of antisense methodology are known in the art, for example see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49.

A “triplex nucleic acid” or “triplex oligonucleotide” is a polynucleotide or oligonucleotide that can bind to a double-stranded DNA in a sequence-specific manner to form a triple-strand helix. Formation of such triple helix structure has been shown to modulate transcription of the targeted gene (Duval-Valentin et al., 1992, Proc. Natl. Acad. Sci. USA, 89, 504).

For further details relating to known techniques and protocols for manipulation of nucleic acid, for example, in preparation of nucleic acid constructs, mutagenesis, sequencing, introduction of DNA into cells and gene expression, and analysis of proteins, see, for example, Current Protocols in Molecular Biology, 5th ed., Ausubel et al. eds., John Wiley & Sons, 2005 and, Molecular Cloning: a Laboratory Manual: 3^{rd} edition Sambrook et al., Cold Spring Harbor Laboratory Press, 2001.

Granulocytes

Granulocytes are a class of leukocytes characterized by prominent cytoplasmic granules. There are three major granulocyte cell types: neutrophils, eosinophils and basophils.

Neutrophils

The most numerous of the granulocytes are the neutrophils which comprise approximately 60% of blood leukocytes. During inflammation the number of neutrophils present in the blood dramatically increases. These cells are highly phagocytic and form the first line of defence against invading pathogens, especially bacteria. They are also involved in the phagocytosis of dead tissue after injury during acute inflammation. Many of the defence mechanisms employed by neutrophils against pathogens, such as the release of granule contents and the generation of reactive oxygen species are pro-inflammatory and damaging to host tissue. In conditions characterized by excessive activation of neutrophils and/or impaired neutrophil apoptosis, chronic or persistent inflammation may result.

Eosinophils

Eosinophils comprise approximately 1-3% of blood leukocytes. Their primary role is in defence against parasites, in particular against helminths and protozoal infection. In this regard, the cells comprise lysosomal granules containing cytotoxic compounds such as eosinophil cation protein, major basic protein, and peroxidase and other lysomal enzymes. Eosinophils are attracted by substances released by activated lymphocytes and mast cells. Although eosinophils may play a role in regulating hypersensitivity reactions by, for example, inhibiting mast cell histamine release degranulation, these cells may also damage tissue in allergic reactions. The cells accumulate in tissues and blood in a number of circumstances, for example, in hayfever, asthma, eczema etc. As a result, through degranulation, they may contribute to or cause tissue damage associated with allergic reactions, for example in asthma or allergic contact dermatitis.

Basophils

Basophils, which comprise less than 1% of circulating leukocytes, have deep blue granules that contain vasoactive substance and heparin. In allergic reactions, they are activated to degranulate, which may cause local tissue reactions and symptoms associated with acute hypersensitivity reactions.

Treatment

“Treatment” or “therapy” includes any regime that can benefit a human or non-human animal. The treatment may be in respect of an existing condition or may be prophylactic (preventative treatment). Treatment may include curative, alleviation or prophylactic effects.

The present invention may be used to treat any disease in which granulocytes contribute to the disease pathology. In one embodiment the disease is a disease in which granulocytes are principally responsible for the disease pathology. Such diseases include, but are not limited to those characterised by leukocytosis, neutrophilia, granulocytosis, or eosinophilia. Such conditions may result in symptoms such as inflammation, allergic reactions, drug reactions, cardiac abnormalities etc. Diseases for which the invention may find use include those mediated by neutrophils, eosinophils, basophils or two or more thereof.

The invention may be used to treat diseases in which modulation of granulocyte, for example neutrophil, activation and/or infiltration may be therapeutically useful. In a particular embodiment of the invention, modulation of neutrophilactivity may be used for the treatment of inflammatory diseases, such as chronic inflammatory diseases, associated with excessive neutrophil infiltration and neutrophil-mediated tissue damage and remodelling. Thus, in one embodiment of the third aspect of the invention, the inflammatory disease is a chronic inflammatory disease. Examples of such chronic inflammatory disease include, but are not limited to vasculitis, pulmonary fibrosis, and ischaemia reperfusion injury. Other inflammatory diseases for which the invention may find use include inflammatory muscle disease, rheumatoid arthritis, allograft rejection, diabetes, multiple sclerosis (MS)/experimental autoimmune encephalomyelitis (EAE), systemic lupus erythematosus (SLE), dermatitis, and asthma, allergies, allergic inflammatory diseases (acute and chronic), parasite pathologies and inflammation associated with obesity. Other neutrophil mediated conditions for which the present invention may find use include, but are not limited to, neutrophil mediated inflammatory conditions such as pleurisy, lung fibrosis, systemic sclerosis and chronic obstructive pulmonary disease (COPD).

The invention may also be used in the treatment of various cancers. “Treatment of cancer” includes treatment of conditions caused by cancerous growth and/or vascularisation and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, prostate , cervical and ovarian carcinoma, non-small cell lung cancer, hepatocellular carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, astrocytomas, gliomas and retinoblastomas.

The invention may be particularly useful in the treatment of existing cancer and in the prevention of the recurrence of cancer after initial treatment or surgery.

In another embodiment of the invention, the granulocyte mediated condition is an eosinophil mediated condition. Eosinophil mediated conditions for which the present invention may find use include, but are not limited to inflammatory lung disease, for example, asthma, atopic dermatitis, NERDS (nodules eosinophilia, rheumatism, dermatitis and swelling), hyper-eosinophilic syndrome or pulmonary fibrosis, contact dermatitis, eczema, hayfever or other allergic reactions. Other conditions, in which eosinophils may be involved and for which the invention may find use include inflammatory bowel disease (IBD), vasculitic granulomatous diseases including polyarteritis and Wegeners granulomatosis, auto-immune diseases, eosinophilic pneumonia, sarcoiditis and idiopathic pulmonary fibrosis.

In a further embodiment of the invention, the granulocyte mediated condition is a basophil mediated condition for example an allergic reaction, such as an acute hypersensitivity reaction. Other basophil mediated conditions for which the present invention may find use include, but are not limited to, asthma and allergies such as hayfever, chronic urticaria, psoriasis, eczema, inflammatory bowel disease, ulcerative colitis, Crohn's disease, COPD (chronic obstructive pulmonary disease) and arthritis.

Pharmaceutical Compositions

Pharmaceutical compositions according to the present invention, and for use in accordance with the present invention may comprise, in addition to active ingredients, e.g. a lactoferrin modulator, a pharmaceutically acceptable excipient, a carrier, buffer stabiliser or other materials well known to those skilled in the art (see, for example, (Remington: the Science and Practice of Pharmacy, 21^{st} edition, Gennaro A R, et al, eds., Lippincott Williams & Wilkins, 2005.). Such materials may include buffers such as acetate, Tris, phosphate, citrate, and other organic acids; antioxidants; preservatives; proteins, such as serum albumin, gelatin, or immunoglobulins; hydrophilic polymers such as polyvinylpyrrolidone; amino acids such as glycine, glutamine, asparagine, histidine, arginine, or lysine; carbohydrates; chelating agents; tonicifiers; and surfactants.

The pharmaceutical compositions may also contain one or more further active compound selected as necessary for the particular indication being treated, preferably with complementary activities that do not adversely affect the activity of the composition of the invention. For example, in the treatment of cancer, in addition to a lactoferrin modulator, such as an anti-lactoferrin antibody the formulation or kit may comprise an additional component, for example a second or further lactoferrin modulator, a chemotherapeutic agent, or an antibody to a target other than lactoferrin, for example to a growth factor which affects the growth of a particular cancer.

The active ingredients (e.g. lactoferrin modulators) may be administered via microspheres, microcapsules liposomes, and other microparticulate delivery systems. For example, active ingredients may be entrapped within microcapsules which may be prepared, for example, by coacervation techniques or by interfacial polymerization, for example, hydroxymethylcellulose or gelatine microcapsules and poly-(methylmethacylate) microcapsules, respectively, in colloidal drug delivery systems (for example, liposomes, albumin microspheres, microemulsions, nano-particles and nanocapsules) or in macroemulsions. For further details, see Remington: the Science and Practice of Pharmacy, 21^{st} edition, Gennaro A R, et al, eds., Lippincott Williams & Wilkins, 2005.

Sustained-release preparations may be used for delivery of active agents. Suitable examples of sustained-release preparations include semi-permeable matrices of solid hydrophobic polymers containing the antibody, which matrices are in the form of shaped articles, e.g. films, suppositories or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and ethyl-L glutamate, non-degradable ethylene-vinyl acetate, degradable lactic acid-glycolic acid copolymers, and poly-D-(−)-3-hydroxybutyric acid.

As described above nucleic acids may also be used in methods of treatment. Nucleic acid for use in the invention may be delivered to cells of interest using any suitable technique known in the art. Nucleic acid (optionally contained in a vector) may be delivered to a patient's cells using in vivo or ex vivo techniques. For in vivo techniques, transfection with viral vectors (such as adenovirus, Herpes simplex I virus, or adeno-associated virus) and lipid-based systems (useful lipids for lipid-mediated transfer of the gene are DOTMA, DOPE and DC-Chol, for example) may be used (see for example, Anderson et al., Science 256: 808-813 (1992). See also WO 93/25673).

In ex vivo techniques, the nucleic acid is introduced into isolated cells of the patient with the modified cells being administered to the patient either directly or, for example, encapsulated within porous membranes which are implanted into the patient (see, e.g. U.S. Pat. Nos. 4,892,538 and 5,283,187). Techniques available for introducing nucleic acids into viable cells may include the use of retroviral vectors, liposomes, electroporation, microinjection, cell fusion, DEAE-dextran, the calcium phosphate precipitation method, etc.

The lactoferrin modulator(s) may be administered in a localised manner to a target site, for example a tumour site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Dose

The lactoferrin modulators for use in the invention are suitably administered to an individual in a “therapeutically effective amount”, this being sufficient to show benefit to the individual. The actual dosage regimen will depend on a number of factors including the condition being treated, its severity, the patient being treated, the agents being used, and will be at the discretion of the physician.

### BRIEF DESCRIPTION OF THE DRAWINGS

The invention will now be described further in the following non-limiting examples. Reference is made to the accompanying drawings in which:

FIG. 1(a) illustrates immunohistochemical detection of neutrophils in Burkitt lymphoma (i) and spleen (positive control) (ii) sections. Insert images represent isotype control

FIG. 1(b) illustrates a graph summarising neutrophil chemotaxis towards increasing concentrations of BL cells in the presence of fMLP (100 nM) n=3; *p<0.05

FIG. 1(c) illustrates a graph summarising fMLP-induced neutrophil chemotaxis BL analysed using cell-conditioned media obtained at the indicated time points n=3; p<0.05

FIG. 1(d) illustrates neutrophil chemotaxis towards fMLP analysed in the presence of control or transfected BL2 cells obtained following a Oh and 5 h incubation at 37° C. Apoptosis levels were assessed by flow cytometry following staining with annexinV/propidium iodide (% apoptosis Oh: BL2 7.53%, BL2/bcl2 3.27%; 5 h: BL2 10.93%, BL2/bcl2 7.41%) All error bars indicate s.e.m.

FIG. 2a) illustrates a graph of fMLP-induced (100 nM) neutrophil chemotaxis towards >50 kDa and <50 kDa fractions of BL medium, fMLP alone (+ve control), assay medium (−ve control) and BL medium (unfiltered+fMLP). Error bars indicate SEM. *p<0.001; compared to the corresponding positive control. Results indicate the mean number of migrated neutrophils counted in ten random high power fields and are representative of three independent experiments.

FIG. 2b) illustrates the results of fMLP-induced (100 nM) neutrophil chemotaxis towards +vely charged fraction (Q1) of the >50 kDa fraction of the BL medium, −vely charged fraction (Q2) of the >50 kDa fraction of the BL medium, fMLP alone (+ve control), assay medium (−ve control) and Q1 and Q2 fraction (unbound and eluant fraction) of serum-free medium (no BL).

FIG. 3(a) illustrates neutrophil chemotaxis in the presence of polyclonal human anti-lactoferrin antibody (grey) or isotype control (black) n=3; *p<0.05 vs. isotype control, NS=non significant vs. fMLP anti-lactoferrin control Error bars indicate s.e.m.

FIG. 3(b) illustrates dose-response analysis of purified human lactoferrin n=3; *p<0.05 Error bars indicate s.e.m.

FIG. 3(c) illustrates neutrophil chemotaxis towards different chemoattractants n=3; *p<0.05 Error bars indicate s.e.m.

FIG. 3(d) illustrates C5a-induced monocyte (i) or macrophage (ii) chemotaxis Error bars indicate s.e.m.

FIG. 3(e) illustrates neutrophil migration in the presence of lactoferrin in the top or bottom compartment of the transwell insert (n=3; NS=non-significant) Error bars indicate s.e.m.

FIG. 3(f) illustrates neutrophil chemotaxis towards lactoferrin or transferrin (n=3; *p<0.05). Error bars indicate s.e.m.

FIG. 3(g) illustrates total cell obtained from peritoneal lavage. (*p<0.05 vs. transferrin). Error bars indicate s.e.m.

FIG. 3(h); illustrates neutrophil number (GR1 positive) obtained from peritoneal lavage.; *p<0.05 vs. thioglycollate control **p<0.01 vs. transferrin control. Error bars indicate s.e.m.

FIG. 4 illustrates neutrophil chemotaxis towards supernatants obtained from MCF7 cells and an isotype control after 5 h incubation in an fMLP-induced in vitro chemotaxis assay (fMLP: 100 nM) Results are representative of three independent experiments. Error bars indicate SEM. *p<0.001 and #p>0.05 compared to fMLP control

FIG. 5 illustrates the results of an in vitro neutrophil chemotaxis assay comparing the inhibitory effect on neutrophil fMLP-induced chemotaxis of lactoferrin from milk (synthesised by mammary cells) or neutrophils (stored in secondary granules). Both types of lactoferrin were added at a concentration of 10 μg/ml. Results represent the mean number of migrated neutrophils counted in ten random high power fields from three independent experiments. Error bars indicate SEM.

FIG. 6(a) illustrates representative flow cytometry overlays illustrating the expression of CD62L assessed in fMLP (100 nM), TNFα (1 ng/ml) or PMA (100 nM)-stimulated neutrophils (30 min at 37° C.) that were pre-incubated (40 min at 37° C.) in the presence or not of lactoferrin (10 ug/ml). Control (middle peak) and stimulated neutrophils (right or left peak for lactoferrin-treated)

FIG. 6(b) is a bar chart illustrating the effect of effect of the expression of CD62L assessed in fMLP (100 nM), TNFα (1 ng/ml) or PMA (100 nM)-stimulated neutrophils (30 min at 37° C.) that were pre-incubated (40 min at 37° C.) in the presence or not of lactoferrin (10 ug/ml), (representative overlays being shown in FIG. 6(a)). n=3; *p<0.05, **p<0.01. All error bars indicate s.e.m.

FIG. 6(c) illustrates representative flow cytometry overlays illustrating the expression of CD11b assessed in fMLP (100 nM), TNFα (1 ng/ml) or PMA (100 nM)-stimulated neutrophils (30 min at 37° C.) that were pre-incubated (40 min at 37° C.) in the presence or not of lactoferrin (10 ug/ml). Control (middle peak) and stimulated neutrophils (right or left peak for lactoferrin-treated)

FIG. 6(d) is a bar chart illustrating the effect of effect of the expression of CD11b assessed in fMLP (100 nM), TNFα (1 ng/ml) or PMA (100 nM)-stimulated neutrophils (30 min at 37° C.) that were pre-incubated (40 min at 37° C.) in the presence or not of lactoferrin (10 ug/ml), (representative overlays being shown in FIG. 6(c)). n=3; *p<0.05, **p<0.01. All error bars indicate s.e.m.

FIG. 6(e) illustrates time-lapse video microscopy of control or lactoferrin pre-treated neutrophils (10 ug/ml; 40 min at 37° C.) stimulated with 1 uM fMLP. Representative images at 30 min time point (i) and quantification (ii) of video microscopy from five different fields; *p<0.05. Error bars indicate s.e.m.

FIG. 7(a) illustrates RT-PCR analysis in several cell lines control (V) or stimulated to undergo apoptosis (A). MCF7-caspase 3 (25.4% apoptosis; 100 uM etoposide, 20 h), Jurkat (18.4% apoptosis; 1 uM staurosporine, 3 h), BL2 (12.46% apoptosis) and BL2/bcl2 (7.42% apoptosis; 1 uM staurosporine, 1 h)

FIG. 7(b) (i) illustrates lactoferrin expression in A549 cells at defined time points (h) following stimulation with 100 uM etoposide or 1 uM staurosporine.

FIG. 7(b) (ii) illustrates the effect of addition of pancaspase inhibitor, zVAD-fmk (100 ug/ml) for 12 h in order to prevent etoposide-induced apoptosis in A549 cells.

FIG. 7(c) illustrates immunoblot analysis of TCA precipitated supernatants from: BL2 and BL2/bcl2 in the presence (+) or absence (−) of staurosporine (1 uM) in serum-free conditions for 1 h. A549 cells were stimulated with (+) or without (−) 100 uM etoposide for 5 h.

FIG. 7(d) illustrates immunoblot analysis where A549 cells were induced to become apoptotic (100 uM etoposide; 20 h) in the presence or absence of brefeldin A (1 ug/ml), a protein release inhibitor.

FIG. 8a illustrates the results of a chemotaxis assay to determine eosinophil migration towards lactoferrin (L) purified from human milk or neutrophils (10 ug/ml) in the presence/absence of eotaxin (EO, 100 nM)

FIG. 8b illustrates the results of a chemotaxis assay to determine eosinophil migration towards varying concentrations of lactoferrin purified from human milk (10 ug/ml) in the presence of eotaxin (100 nM)

FIG. 8c illustrates the results of a chemotaxis assay to determine eosinophil migration towards human lactoferrin (LF) or transferrin (TRF, 10 ug/ml) in the presence/absence of eotaxin (EO, 100 nM)

FIG. 9a illustrates proliferation of Burkitt lymphoma BL2 cells (total cell population) cultured over a 48 h time course in the presence of monoclonal anti-human lactoferrin antibody (mAb) or isotype control (iso).

FIG. 9b illustrates proliferation of Burkitt lymphoma BL2 cells (viable cell population only) cultured over a 48 h time course in the presence of monoclonal anti-human lactoferrin antibody (mAb) orisotype control (iso).

FIG. 10a illustrates the nucleic acid sequence of the gene encoding lactoferrin;

FIG. 10b illustrates the amino acid sequence of the lactoferrin protein;

FIG. 11 shows that Lactoferrin specifically inhibits eosinophil chemotaxis. (A) Chemotaxis assay to determine eosinophil migration towards milk- or neutrophil-derived lactoferrin (LTF −10 μg/ml) in the presence of eotaxin (100 nM) as a chemoattractant. (B) Eosinophil chemotaxis towards varying concentrations of purified human lactoferrin and (C) towards different chemoattractants (fMLP −100 nM, C5a −6.25 ng/ml and LTB₄ −50 nM) (D) Chemotaxis assay in the presence of lactoferrin (10 μg/ml) in the top or bottom compartment of the Transwell insert. All results are representative of the mean of three independent experiments, error bars indicate SEM, and one-way ANOVA was performed followed by Bonferroni post-hoc test. *P<0.05 NS=non-significant.

FIG. 12 shows that inhibition of eosinophil chemotaxis by lactoferrin occurs irrespective of the iron-saturation status and iron-binding properties of lactoferrin. (A) Chemotaxis assays to determine eosinophil migration towards eotaxin in the presence of recombinant iron-depleted (Apo-), partially-iron saturated and fully iron-saturated (Holo-) recombinant lactoferrin (10 μg/ml) (B) Eosinophil chemotaxis towards eotaxin in the presence of recombinant purified human lactoferrin (LTF-10 μg/ml) and purified human transferrin (TF-10 μg/ml). All results are representative of the mean of three independent experiments, error bars indicate SEM, and one-way ANOVA was performed followed by Bonferroni post-hoc test *P<0.05 NS=non-significant.

### EXAMPLES

Methods

Cell Isolation

Mononuclear and polymorphonuclear (PMN) leukocytes were isolated from peripheral venous blood as previously described Dransfield et al 1995, Blood 85, 3264 Neutrophils represented >95% of isolated PMN cells. Eosinophil isolation was according to the method as described in Rossi et al (1998) J. Clin. Invest. 101, 2869-2874. Monocytes (>90% CD14⁺ cells) were positively selected from isolated mononuclear leukocytes using CD14 magnetic beads (Miltenyi Biotec). Human monocyte-derived macrophages were obtained following culture of monocytes for six days in IMDM+10% autologous serum.

Peritonitis Model

Mice (8 to 12 week old female C57BU6 mice, n=7 per group) were injected i.p. with purified human lactoferrin or transferrin (Sigma Aldrich; 500 ng in saline/0.1% BSA) or saline/0.1% BSA alone followed by a second i.p. injection with 1% thioglycollate (500 μl) or saline/0.1%BSA after 20 min. Recruited leukocytes were harvested after 4 h by peritoneal lavage with ice-cold saline containing 2 mM EDTA. Harvested cells were counted using nucleocounter (Nucleocounter Chemometec), excluding in this way any non-nucleated cells (red blood cells). To determine the number of neutrophils (GR1⁺), cells were counted by cell counting beads (Beckman Coulter) and immunolabelled with PE-conjugated anti-mouse Ly6-GR1. Cytospin samples were also prepared.

Histology and Immunohistochemistry

Six to 10-week old Balb/c SCID mice were injected i.p. with 10⁷ BL2 cells. Tumours developed i.p. within 2 months of injection. Mice were sacrificed and tumours excised. For positive control, Balb/c mice were immunised with sheep red blood cells and spleens harvested and frozen 7 days after i.p. injection. Immunohistochemistry was performed on frozen acetone-fixed sections (5 μm) of BL or spleen tissues using biotinylated anti-mouse GR1 antibody (10 μg/ml; Biolegend) or isotype control (Serotec). Non-specific adsorption of antibodies was blocked using serum-free Protein Block (Dako Cytomation). Reactions were amplified using Vectastain Elite ABC avidin-biotinylated peroxidase complexes. Hematoxylin was used as counterstain.

Chemotaxis Assay

In vitro leukocyte chemotaxis was measured according to Truman et al. using polyvinyl uncoated Transwell inserts (Costar, 5 μm pore size). Chemotactic agents included fMLP (100 nM; Sigma Aldrich), C5a (6.25 ng ml⁻¹; Sigma Aldrich), IL-8 (50 nM; R&D Systems) LTB₄ (100 nM; Sigma Aldrich) and human eotaxin (100 nM; PeproTech). Incubation time (37° C.; 5% CO₂) varied for cell type (neutrophils and eosinophils: 60 min; monocytes: 90 min; macrophages: 4 h). Unless otherwise stated, lactoferrin was used at 10 ug/ml concentration. For neutralisation experiments, polyclonal rabbit anti-human lactoferrin antibody (Sigma) and negative control rabbit immunoglobulin (Dako Cytomation) were used. Filters were observed using an inverted microscope (Axiovert 25 Zeiss) and relative cell migration was determined by measuring the number of migrated cells in ten random high-power fields (400× magnification).

Size Fractionation and Ion Exchange Chromatography

Size fractionation of BL2 cell conditioned media was performed using filters with specific molecular weight cut-off sizes (Amicon Centrifugal filters YM-50 and YM-100, Millipore), following manufacturer's instructions. Ion exchange chromatography was carried out using Sepharose Fast Flow beads (Sigma Aldrich) with either positive (Q beads) or negative (S beads) charge. Prior to use, beads were washed with PBS and neutralising buffer (10 mM Tris; pH 7.0). BL2 cell conditioned medium or control medium (RPMI 1640) was mixed with the beads and incubated at room temperature for 5 min. Samples were centrifuged (300 g, 5 min) and supernatants stored. Beads were washed with neutralising buffer and bound proteins were then eluted by adding the corresponding elution buffer (for S beads: 10 mM Tris, 0.5M NaCl; pH 10; for Q beads: 10 mM NaAc, 0.5 M NaCl; pH 4). Following a 5-min incubation at room temperature, beads were centrifuged (300 g, 5 min) and supernatants collected and analysed. Prior to chemotaxis analysis, the supernatants were diluted (1:100) and pH was adjusted to 7.0. Proteins were identified by peptide mass fingerprinting using MALDI mass spectrometry. Process carried out by SIRCAMS, School of Chemistry, University of Edinburgh.

Flow Cytometry

Unless otherwise stated, cells were suspended in PBS containing 5% normal mouse serum or 0.1% BSA and all antibody incubations were performed for 20 min on ice. Mouse neutrophils were defined based on the expression of GR1 epitope using PE-conjugated rat anti-mouse Ly6G (GR1, eBioscience). For the assessment of neutrophil activation, the following antibodies were used: FITC-conjugated anti-CD62L (FMC46, mlgG2b, AbD Serotec) and APC-conjugated anti-CD11b (ICRF44, mlgG1, BD Pharmingen). Isotype controls included mouse IgG1:FITC (AbD Serotec), mouse IgG1:APC (BD Pharmigen) and rat IgG2b:PE (eBioscience). Cell apoptosis was determined following labelling with annexin V/propidium iodide. Samples were analysed using a BD FACS Calibur or FACScan cytometer (BD Biosciences) and data were analysed using BD CellQuest software.

Reverse-Transcription (RT-PCR) Analysis

Total RNA was extracted from cells using Qiagen RNeasy kit, according to the manufacturer's instructions. Total RNA (2 μg) was reverse-transcribed using the SuperScript III RT kit (Invitrogen), according to protocol. Resulting cDNAs were used as template in PCR experiments at a concentration of 1 ng/50 μl of PCR mixture. The primers used were: forward LTF (5′-TGTCTTCCTCGTCCTGCTGTTCCTCG-3′) and reverse LTF (5′-CTGCCTCGTATATGAAACCACCATCAA-3′), forward GAPDH primer (5′-CGACAGTCAGCCGCATCTTCTTTTGCGTCG-3′) and reverse GAPDH primer (5′-GGACTGTGGTCATGAGTCCTTCCACGATAC-3′). Purified PCR products (QIAquick gel extraction kit (Qiagen)) were sequenced to confirm validity by the Sequencing Service (School of Life Sciences, University of Dundee, UK) using Applied Biosystems Big-Dye 3.1 chemistry on an Applied Biosystems model 3730 automated capillary DNA sequences.

Immunoblotting

Conditioned media from viable and apoptotic BL2 and A549 cells were collected and their protein content was TCA precipitated. Briefly, 100 μl of TCA were added in 1 ml of conditioned medium at 4 oC. Samples were centrifuged at 18000 g and the pellets washed in ice-cold acetone before re-suspension in sample buffer (NuPAGE, Invitrogen). Samples were resolved by SDS-PAGE using 4-12% Bis-Tris gels (NuPAGE, Invitrogen). Proteins were then electroblotted onto nitrocellulose membrane (NuPAGE, Invitrogen), blocked with 0.5% BSA, probed with monoclonal mouse anti-human lactoferrin (1:100; LF.2B8, AbD Serotec) followed by HRP-conjugated goat anti-mouse IgG (1:2000; Amersham) and visualised using enhanced chemiluminescence (GE Healthcare).

Statistical Analysis

Results from multiple experiments are presented as mean ±standard error of the mean (s.e.m.). One-way analysis of variance (ANOVA) was performed followed by Bonferroni post-hoc test. In all cases, p-values 0.05 were considered to be statistically significant.

### Example 1

### Apoptotic Lymphoma Cells Release a Factor that Prevents Neutrophil Migration

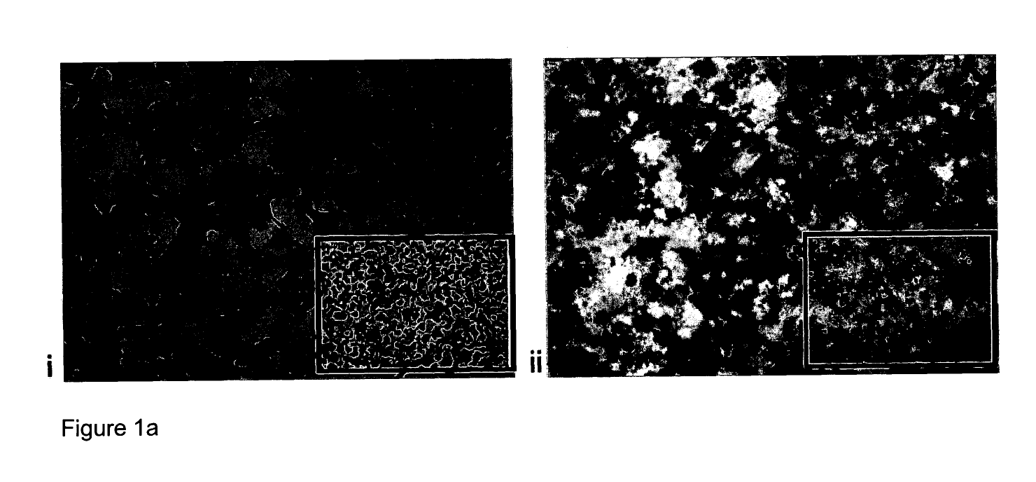

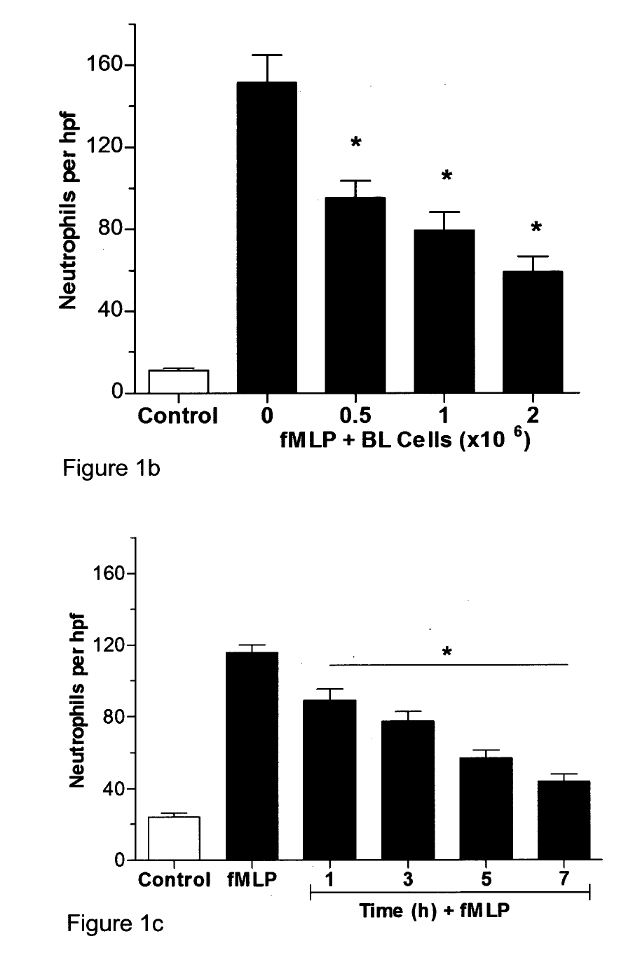

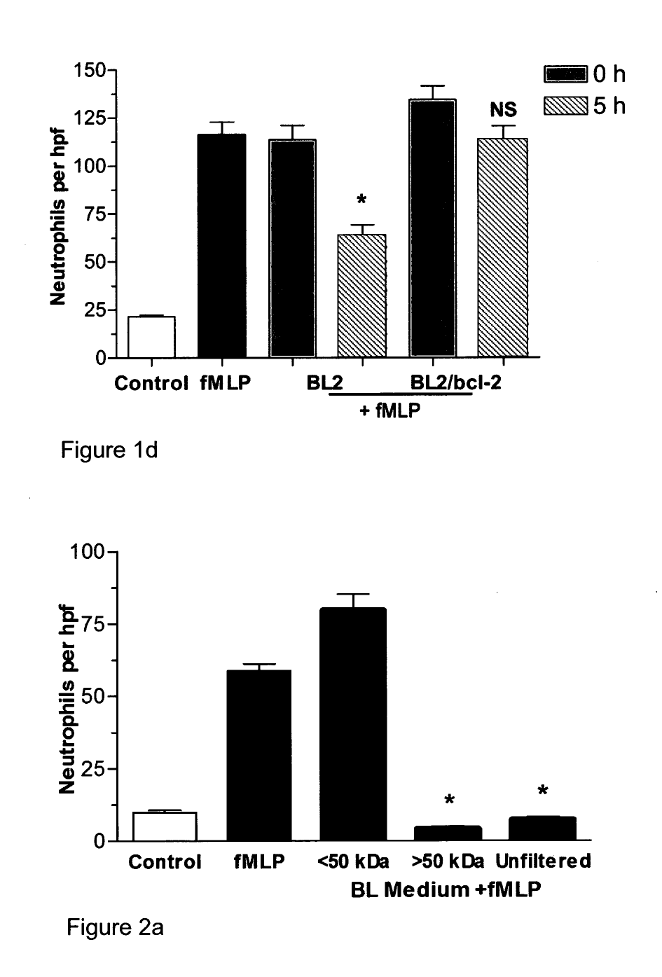

The inventors postulated that the factors released by apoptotic cells include negative regulators of neutrophil chemoattraction and that such factors may also act to limit neutrophil migration to sites of inflammation. The inventors initially analysed Burkitt lymphoma (BL) as a model tissue since this tumour displays high levels of apoptosis and, as is characteristic of all sites displaying high rates of apoptosis, marked infiltration by macrophages that engulf the apoptotic cells, giving rise to the typical ‘starry sky’ histological appearance of this tumour. While macrophages are in abundance, however, neutrophils are absent (FIG. 1a). The inventors next assessed the effects of BL cells on the migratory activity of neutrophils in vitro. Using a Boyden-type chemotaxis assay in which neutrophils were added on top of a transwell filter and were induced under the influence of fMLP to migrate towards the lower chamber containing BL cells, The inventors observed significant inhibition of neutrophil migration in a BL cell concentration-dependent manner (FIG. 1b). This effect was not limited to fMLP-induced neutrophil migration, but was observed irrespective of the chemoattractant used (inhibition in neutrophil migration in C5a-, IL-8- and LTB4-induced chemotaxis). Subsequent chemotaxis assays showed that BL cells actively released an inhibitory factor(s) as BL-conditioned media over a 7-hour time course retained the neutrophil migration inhibitory effect (FIG. 1c). The release of the inhibitory factor appeared to be linked to the levels of apoptosis in the BL cell populations, since the inhibitory activity was lower in BL-conditioned media derived from cells that had been transfected with the suppressor of apoptosis, bcl-2, as compared to parental cells (FIG. 1d).

### Example 2

### Identification of Lactoferrin as Inhibitory Factor

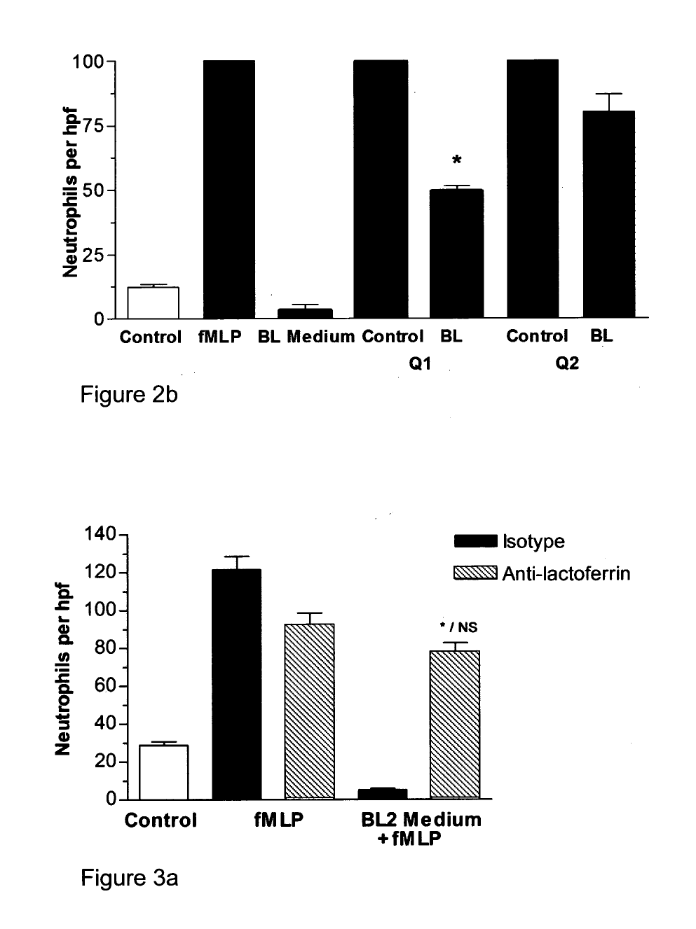

The molecular weight of the inhibitory factor(s) was determined. Briefly, BL2-conditioned media (24 h at 37° C.) were fractionated using filters with specific molecular weight cut-off sizes. Using 50 kDa filter, the >50 kDa and <50 kDa fractions of the BL medium were collected and fMLP-induced (100 nM) neutrophil chemotaxis was assessed. As control, fMLP alone (+ve control), assay medium (−ve control) and BL medium (unfiltered+fMLP) were included. The results are shown in FIG. 2a and show that the inhibitory factor(s) have a molecular weight of over 50 kDa.

Ion exchange analysis was used to determine the μl of the inhibitory factor(s), the results being illustrated in FIG. 2b. Q (+ve charge) Sepharose beads were used in order to distinguish positive and negatively charged molecules in the >50 kDa fraction of the BL medium. BL medium was complexed with positively charged beads (Q beads) to allow negatively charged molecules to become bound to bead surface. Unbound molecules (Q1 fraction; +vely charged) were collected, whereas bound molecules were eluted from the beads (Q2 fraction; −vely charged). Neutrophil migration towards these fractions in the presence of fMLP (100 nM) was assessed. As controls, fMLP alone (+ve control), assay medium (−ve control) and Q1 and Q2 fraction (unbound and eluant fraction) of serum-free medium (no BL) containing 100 nM fMLP were used.

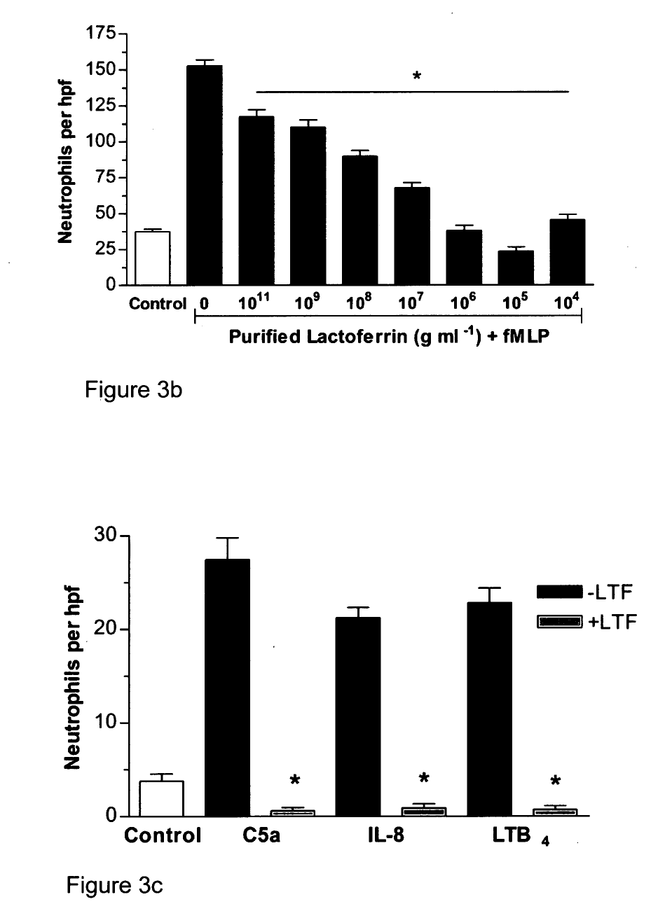

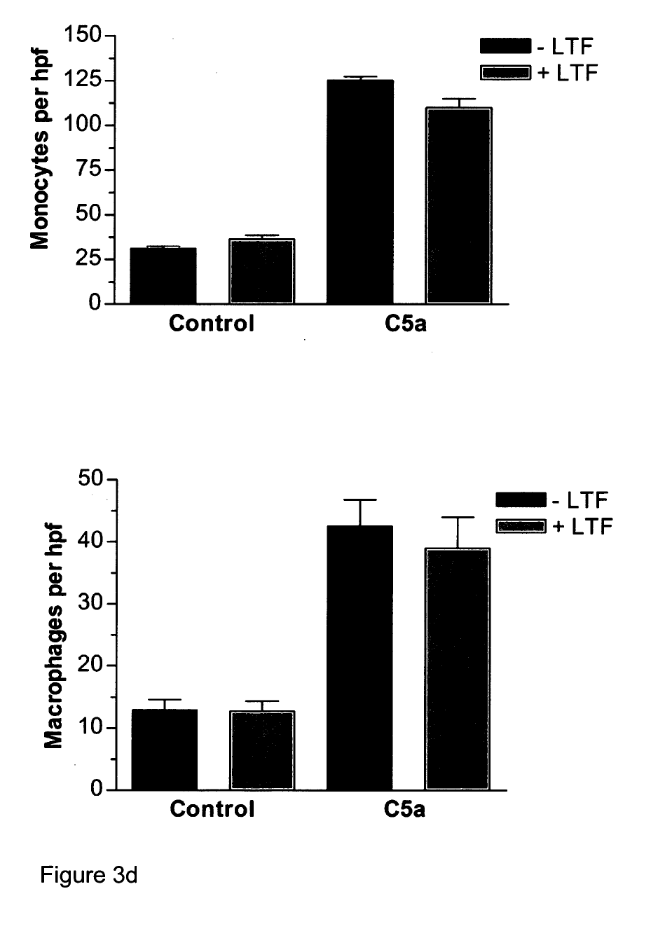

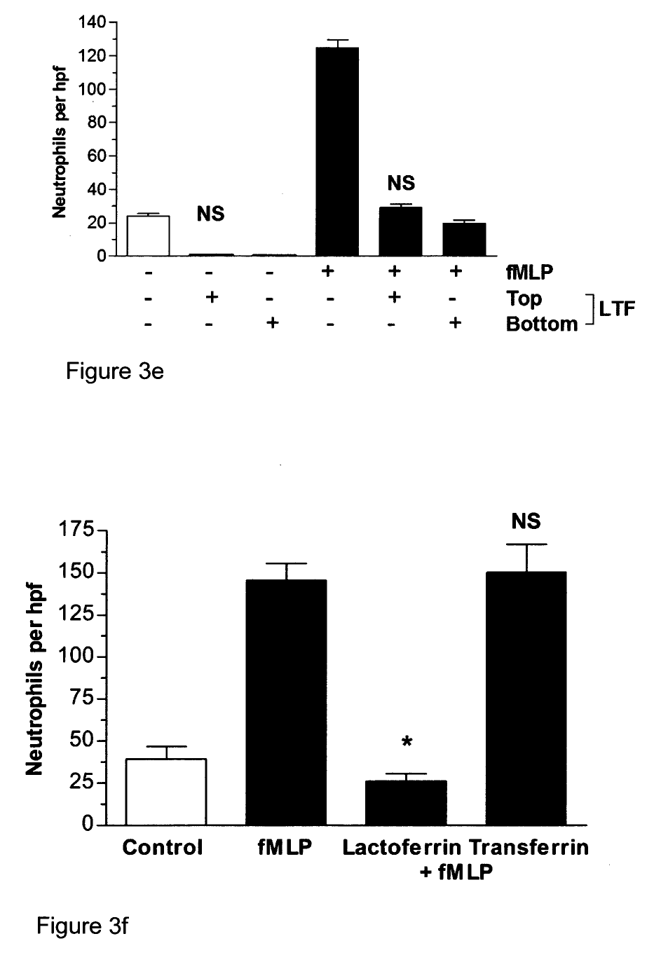

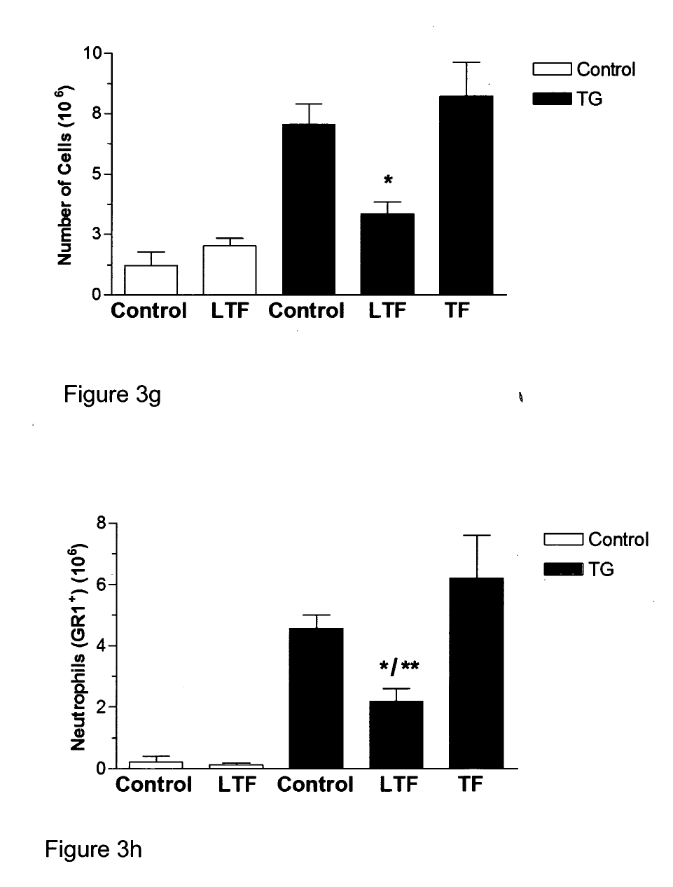

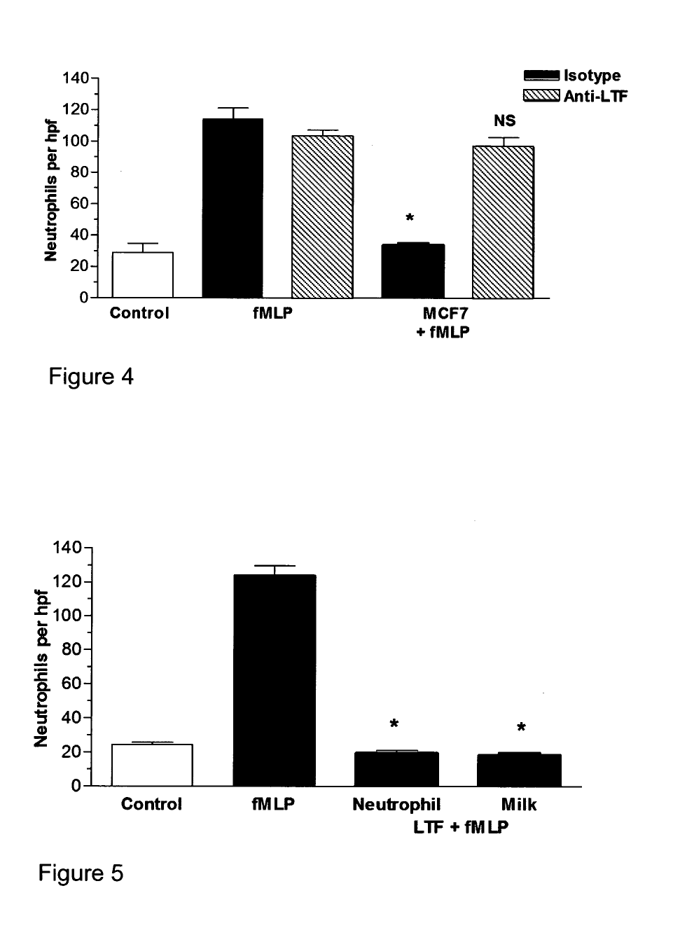

Using the biochemical analyses of BL-conditioned media including the determination of the molecular weight of the inhibitory factor(s), ion exchange chromatography, fingerprinting of the proteins released by BL cells as well as a candidate analysis, the inventors identified the factor released by BL cells that prevented neutrophil chemotaxis as lactoferrin. Lactoferrin is an 80 kDa glycoprotein that belongs to the transferrin family of proteins due to its iron-binding properties. It is a well-characterised component of neutrophil secondary granules, tears, colostrum, saliva and mucus secretions, in which it confers anti-bacterial activity. The inventors observed that addition of anti-lactoferrin antibody to BL-conditioned medium neutralised its neutrophil migration inhibitory activity (FIG. 3a). Similar results were obtained with supernatants obtained from MCF7 cells 9 FIG. 4). Briefly, anti-lactoferrin antibody was shown to abrogate the inhibitory effect on neutrophil chemotaxis towards supernatants obtained from MCF7 cells after 5 h incubation in an fMLP-induced in vitro chemotaxis assay (fMLP: 100 nM). An isotype control was included that failed to present an analogous abrogation of the inhibitory effect, as seen with the anti-lactoferrin antibody. The results indicate that the neutrophil-inhibitory activity is not restricted to BL cell-derived lactoferrin.

Furthermore, lactoferrin purified from human milk displayed dose-dependent inhibitory activity toward neutrophil migration in response to fMLP (FIG. 3b) and also inhibited migration towards C5a, IL-8 and LTB4 to similar levels (FIG. 3c). The neutrophil migration-inhibitory effect was also displayed by lactoferrin purified from human neutrophils (FIG. 5), showing that lactoferrin exerts an inhibitory effect on neutrophil migration irrespective of source of purification. Notably, lactoferrin exerted no toxic effects on neutrophil viability, as assessed by annexinV/propidium iodide staining of control neutrophils and neutrophils pretreated with lactoferrin (>98% viable cells).

### Example 3

### Specificity of Migration Inhibitory Effect of Lactoferrin

To determine whether, amongst professional phagocytes, the migration-inhibitory effects of lactoferrin were specific to neutrophils, The inventors analysed its effects on monocyte and macrophage migration in vitro. As shown in FIG. 3c, chemotaxis of mononuclear phagocytes was unimpaired by lactoferrin. The inventors further assessed whether lactoferrin acted by inhibiting neutrophil migration or promoting neutrophil repulsion. In chemotaxis assays, in which The inventors added lactoferrin to the upper chamber along with neutrophils. The inventors observed inhibition of neutrophil migration towards fMLP and control medium (FIG. 3e), suggesting that lactoferrin exerts a direct effect on neutrophils by inhibiting their migratory ability and not by forcing them to migrate in all directions away from it. As lactoferrin belongs to the transferrin family of iron-binding proteins. The inventors further examined whether its homologous cationic glycoprotein, transferrin, (44% sequence homology) possessed the same neutrophil migration-inhibitory properties: transferrin displayed no such effect (FIG. 3f).

### Example 4

### In Vivo Effects of Lactoferrin

Having established the inhibitory effects of lactoferrin in neutrophil chemotaxis in vitro, the inventors then used a murine peritonitis model to assess the effect of lactoferrin on leukocyte recruitment in vivo. Lactoferrin and transferrin were tested for their ability to affect thioglycollate-induced leukocyte recruitment to the peritoneal cavity. As shown in FIG. 3g-h, thioglycollate caused a rapid recruitment of leukocytes compared with vehicle alone and the recruited leukocytes were predominantly neutrophils (88%). In the presence of lactoferrin, the total number of neutrophils recruited to the peritoneal cavity was reduced by 52.19% compared to control, whereas transferrin had no effect. Lactoferrin did not alter the types of leukocytes recruited by thioglycollate, but rather reduced specifically the proportion and number of neutrophils migrating into the cavity. Thus, similar to its effect on neutrophil chemoattraction in vitro, lactoferrin is a potent inhibitor of neutrophil migration in vivo.

### Example 5

### Effect of Lactoferrin on Neutrophil Activation

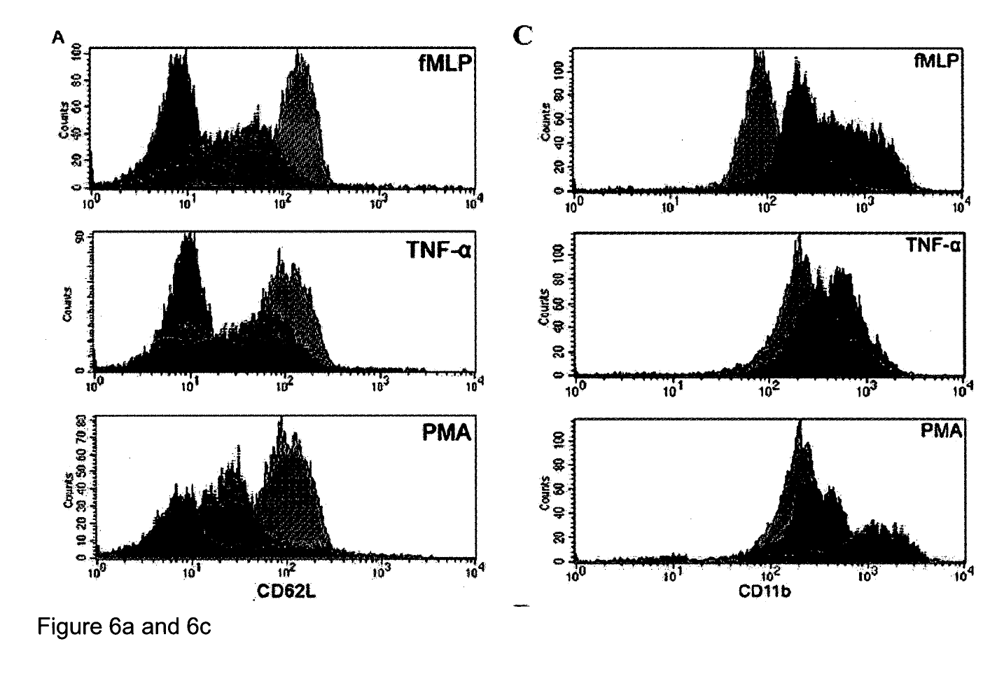

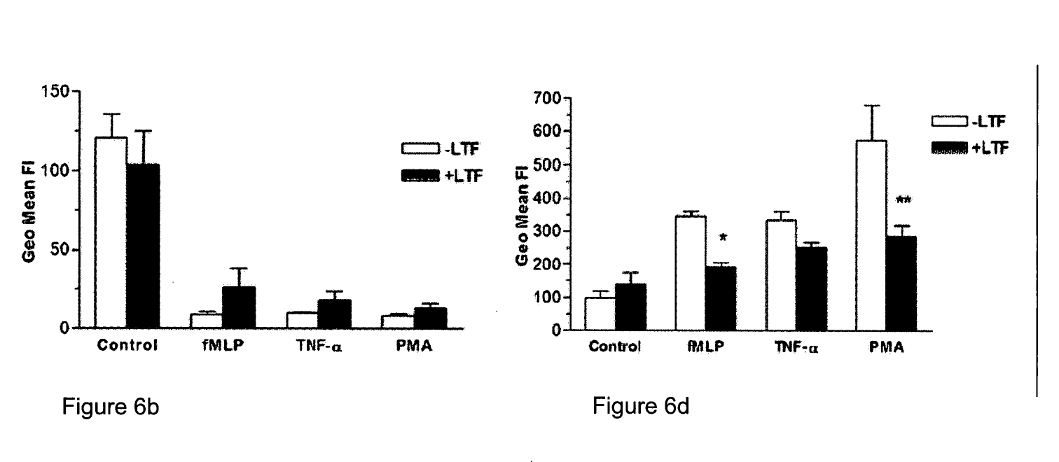

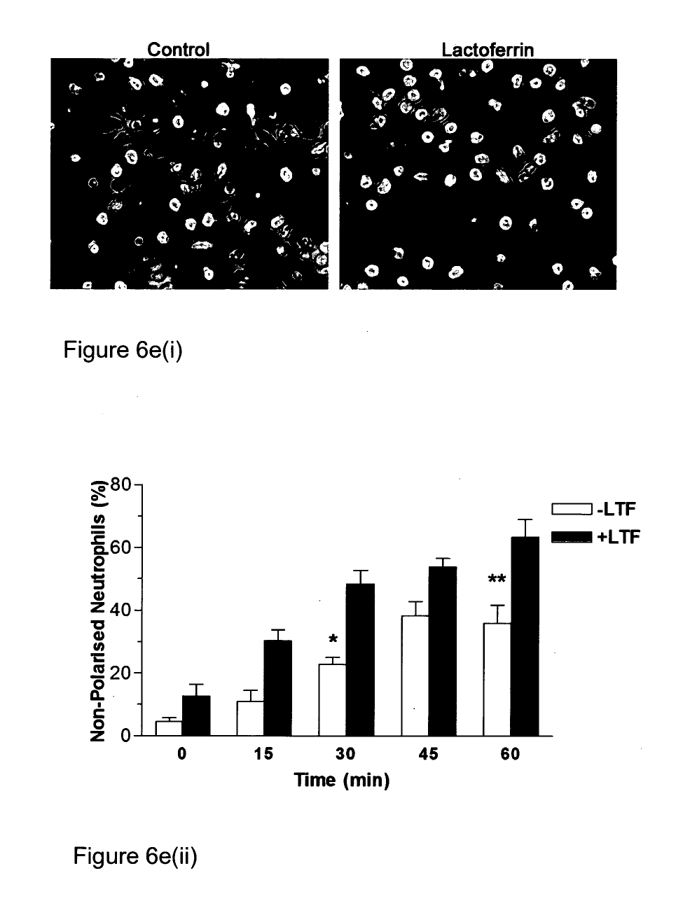

Because neutrophil migration is a multi-step process involving cell activation and polarisation, the inventors reasoned that the observed neutrophil migration-inhibitory effects of lactoferrin might be manifest in the neutrophil activation state. The inventors selected to measure the expression of two known activation-associated markers, CD62L (L-selectin) and CD11b using two-colour flow cytometry. Upon activation, CD62L is cleaved from the neutrophil surface whereas CD11b expression is upregulated as it becomes translocated from intracellular pools to the cell membrane. As shown in FIG. 6a-b, in response to various activation stimuli, including fMLP, TNF-α and PMA, neutrophils pre-treated with lactoferrin displayed lower levels of CD11b and higher levels of CD62L than controls. Subsequent time-lapse video-microscopy analysis of lactoferrin-treated neutrophils was also performed in an attempt to examine any effects of lactoferrin on neutrophil morphology. During a one-hour time course, lactoferrin-pretreated neutrophil populations stimulated with fMLP, displayed greater proportion of non-adherent cells and cells presenting a rounded, non-activated morphology (FIG. 6c). Collectively, these findings extended the observed inhibitory effect of lactoferrin on neutrophil migration and indicated that this effect might be attributed to impairment of neutrophil activation.

### Example 6

### Induction of Apoptosis Upregulates Lactoferrin Expression and Release

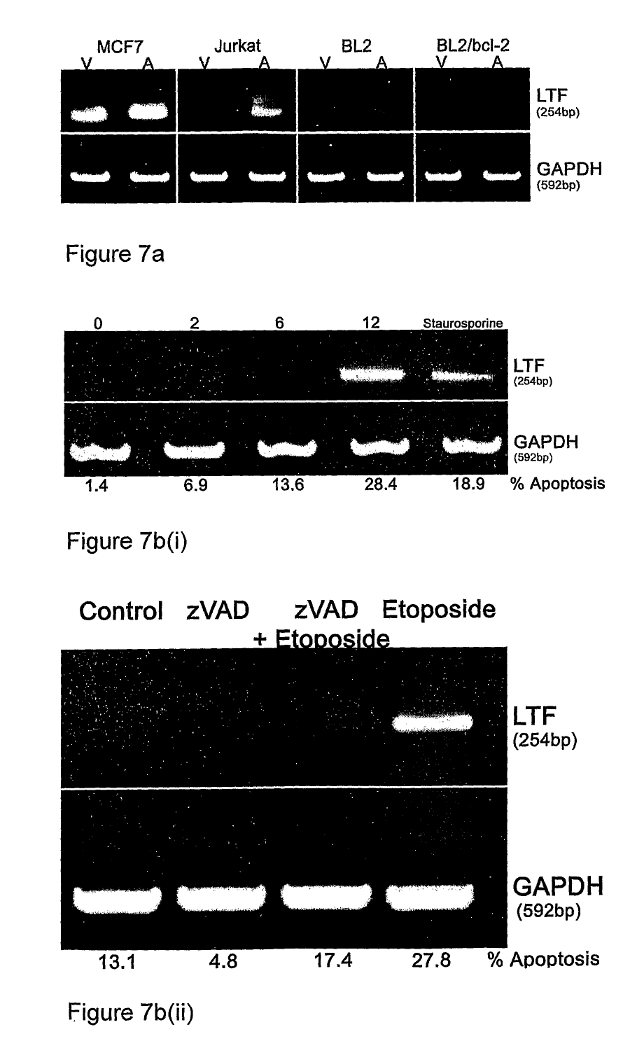

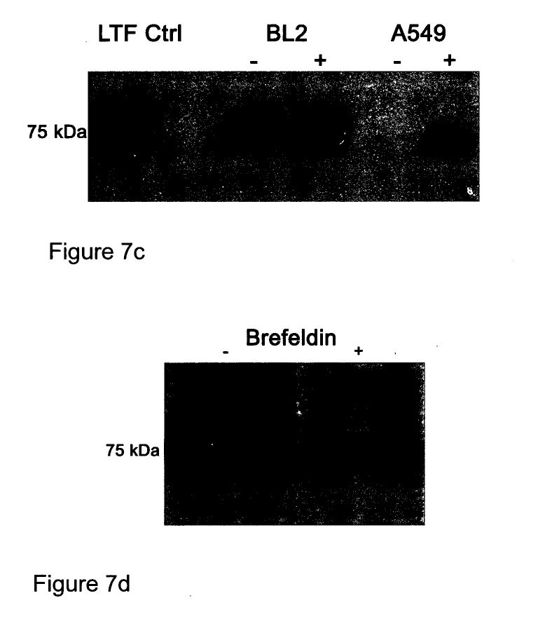

Pursuing the inventors' initial hypothesis that was strengthened by early observations that inhibition of neutrophil migration by BL cells appeared to be correlated with BL-cell apoptosis (FIG. 1d). The inventors assessed lactoferrin expression following induction of apoptosis in a panel of cell types. By transcriptional analysis using semi-quantitative RT-PCR. The inventors found that lactoferrin was expressed, as reported previously, by MCF7 mammary epithelial cells in their viable state but not by Jurkat, BL2 or A549 cells. Upon apoptosis induction, lactoferrin expression was upregulated in MCF7 cells and expressed de novo in Jurkat, BL2 and A549 (FIG. 7). More specifically, lactoferrin was de novo transcribed early after apoptosis-stimulation of A549 cells by etoposide or staurosporine (FIG. 7b). Reduced levels of apoptosis-triggered lactoferrin were evident when A549 cells were treated with the broad caspase inhibitor zVAD-fmk that prevented apoptosis induction. The coupling of lactoferrin production to the induction of apoptosis was further supported by the effects of the apoptosis-inhibitor, Bcl-2; BL cells expressing exogenous Bcl-2 that provided protection from apoptosis, expressed lower levels of lactoferrin upon exposure to the apoptosis inducer, staurosporine than their parental counterparts (FIG. 7a,c). Not only was apoptosis-related lactoferrin production demonstrated at the transcriptional level, lactoferrin protein was recovered from supernatants of cells undergoing apoptosis (FIG. 7c). Treatment of A549 cells with brefeldin, which interferes with intracellular transport of newly synthesised proteins, resulted in inhibition of apoptosis-induced lactoferrin release, further proving the de novo synthesis and secretion of lactoferrin by cells undergoing apoptosis (FIG. 7d). These results demonstrate that lactoferrin is produced and released from cells as a consequence of engagement of their apoptosis program.

### Example 7

### Lactoferrin Inhibits Eosinophil Migration

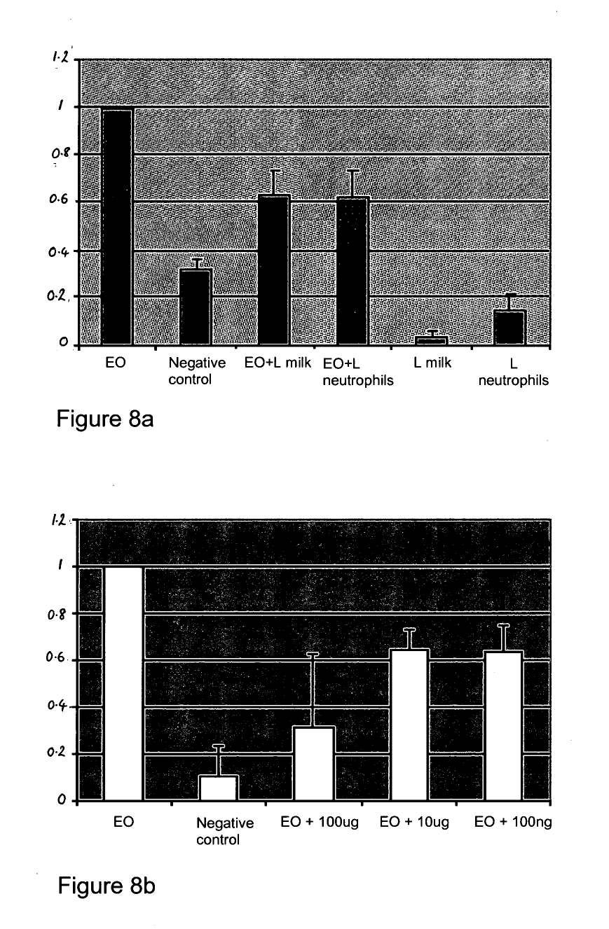

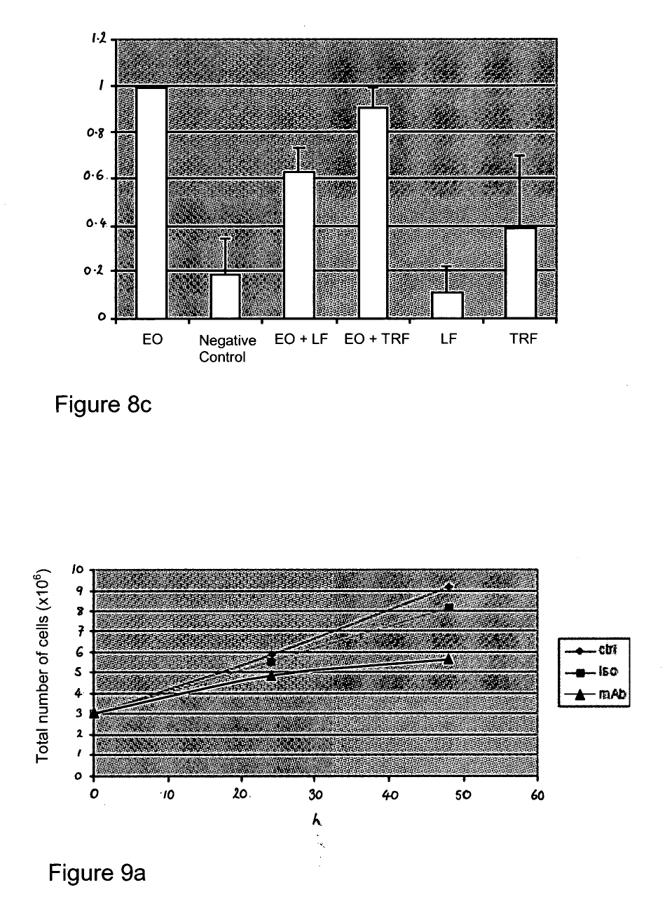

FIG. 8a illustrates the results of a chemotaxis assay to determine eosinophil migration towards lactoferrin (L) purified from human milk or neutrophils (10 ug/ml) in the presence/absence of eotaxin (EO, 100 nM) FIG. 8b illustrates the results of a chemotaxis assay to determine eosinophil migration towards varying concentrations of lactoferrin purified from human milk (10 ug/ml) in the presence of eotaxin (100 nM). The results show that lactoferrin, as well as inhibiting neutrophil migration, also inhibits migration of other granulocytes. FIG. 8c illustrates the results of a chemotaxis assay to determine eosinophil migration towards human lactoferrin (LF) or transferrin (TRF, 10 ug/ml) in the presence/absence of eotaxin (EO, 100 nM) and shows that transferrin does not have the same effct as lactoferrin.

### Example 8

### Endogenous Lactoferrin Supports Growth of Tumour Cells

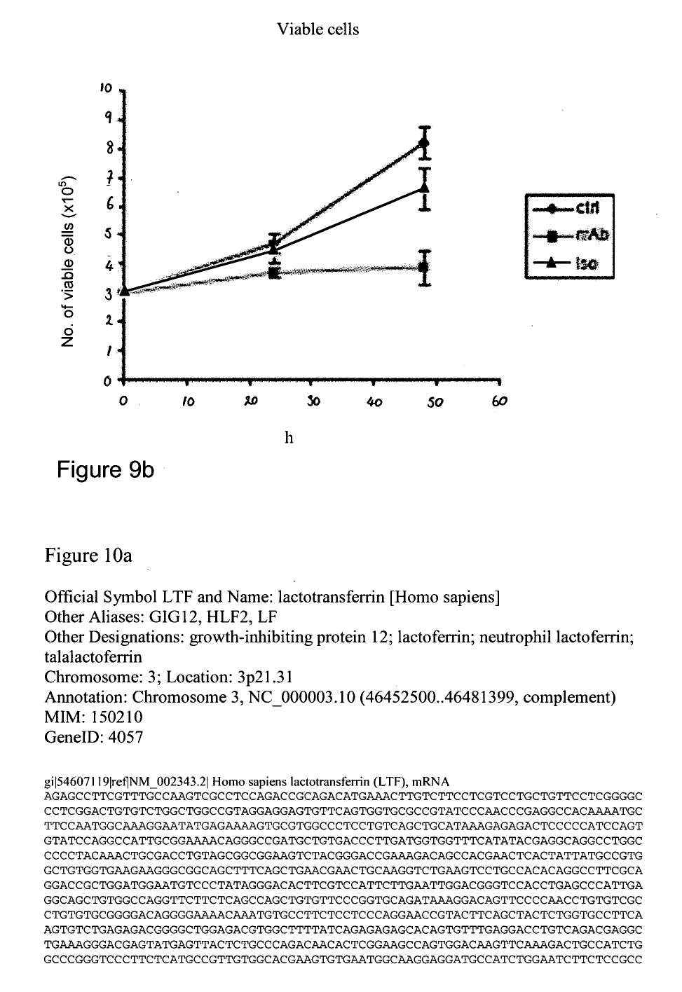

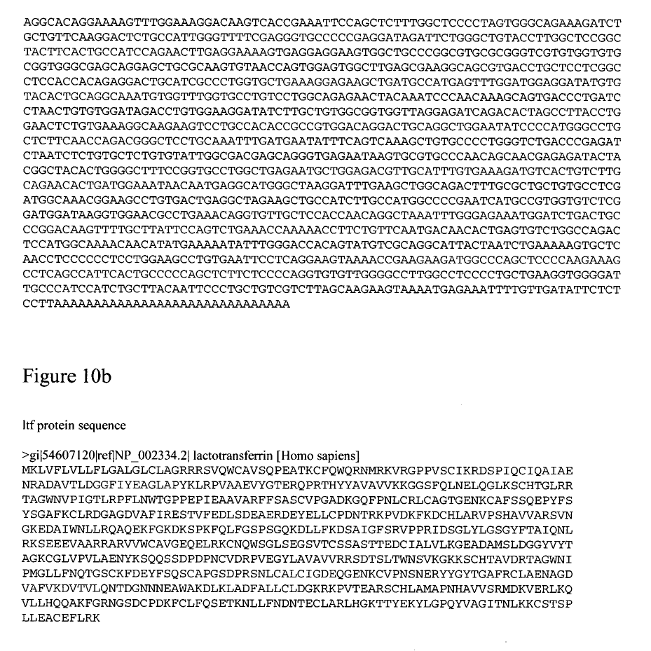

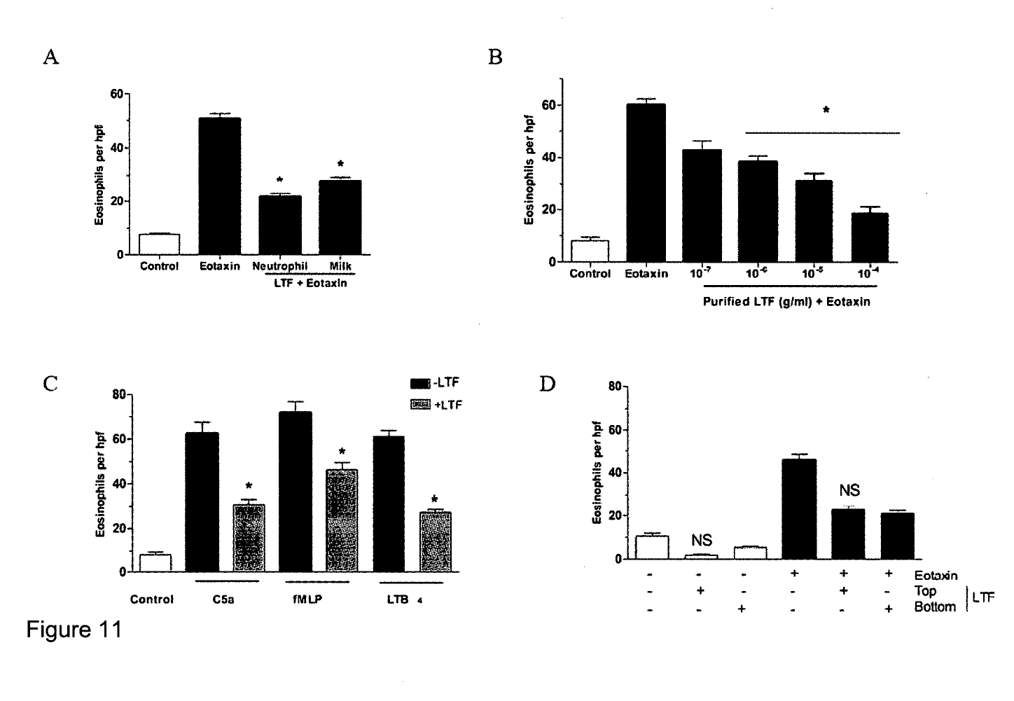

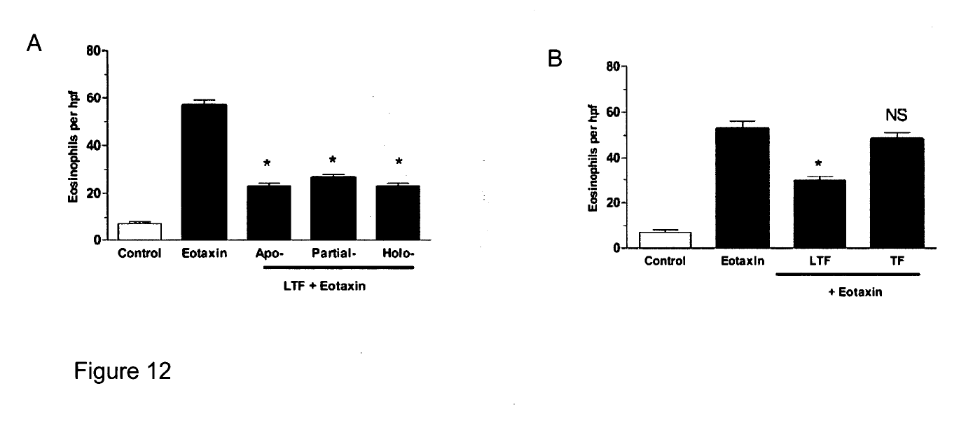

To investigate the effect of lactoferrin on proliferation of cancer cells, Burkitt lymphoma BL2 cells were cultured over a 48 h time course in the presence of monoclonal anti-human lactoferrin antibody (mAb) or isotype control (iso). The results are shown in FIG. 9. FIG. 9a shows that BL2 cells (total cell population) cultured in the absence of lactoferrin display a decreased rate of proliferation. FIG. 9b illustrates proliferation of Burkitt lymphoma BL2 cells (viable cell population only) cultured over the 48 h time course in the presence of monoclonal anti-human lactoferrin antibody (mAb) orisotype control (iso). BL2 cells cultured in the absence of lactoferrin display a decreased rate of proliferation. These results support the use of inhibitors of lactoferrin in anti cancer therapy.

Collectively, the inventors' findings demonstrate novel immunoregulatory and homeostatic functions for lactoferrin, a protein that is well known for its pleiotropic activities that include bacteriostasis, immunomodulation, cell growth regulation and proteolysis. The production of lactoferrin by mammary epithelial cells that secrete the protein during lactation and its constitutive presence in the secondary granules of neutrophils are well established.

Here the inventors demonstrate that lactoferrin is much more generally expressed than previously realised, being linked to a fundamental cellular program, apoptosis, in which it functions to repress acute inflammatory responses to cells undergoing programmed cell death through suppression of neutrophil chemoattraction to apoptotic loci, thereby contributing to the non-phlogistic nature of the apoptosis program.

Lactoferrin can now be counted as one of the few molecules, alongside lipoxins, that negatively regulate neutrophil migration. More importantly, based on the high specificity of its migration-inhibitory properties to neutrophils, lactoferrin is identified here as a promising, therapeutic target for a range of chronic inflammatory conditions—including vasculitis, pulmonary fibrosis and ischaemia reperfusion injury—that are characterised by excessive neutrophil infiltration leading to neutrophil-mediated host tissue damage and remodelling. Furthermore, in certain tumours, in which neutrophils may play a supportive role, limitation of neutrophil infiltration through lactoferrin administration could be therapeutically beneficial. Indeed, lactoferrin has been described as having anti-tumour activity in certain cases. In the majority of tumours, however, from which neutrophils are absent. The inventors propose that, given the well-known oncolytic effects of neutrophils, encouragement of neutrophil infiltration through inhibition of lactoferrin would effect tumour destruction.

### Example 9

Further data relevant to this invention can be found in Bournazou et al., Journal of Clinical Investigation (2009: Full reference Bournazou, I., Pound, J. D., Duffin, R., Bournazos, S., Melville, L. A., Brown, S. B., Rossi, A. G., and Gregory, C. D. (2009). Apoptotic human cells inhibit migration of granulocytes via release of lactoferrin. J Clin Invest 119, 20-32). All references to Figures in this section of the specification are references to Figures that appear in the Bournazou, 2009, paper.

In particular, FIG. 1B of this paper provides images of neutrophils migrating through transwell filters towards fMLP and inhibition by supernatants of tumour cells. In addition, FIG. 2C shows the results of a chemotaxis assay of neutrophils towards BL conditioned medium that was heat-inactivated (90° C. for 10 min).

FIG. 2D shows a MALDI-TOF mass spectrum for the tryptic digest of the peptide band identified as lactoferrin. In addition, neutrophil chemotaxis in the presence of human anti-lactoferrin polyclonal antibody using conditioned media from MCF7 cells is shown in FIG. 3B of Bournazou's paper.

RT-PCR analysis to assess lactoferrin expression by BL cells stably expressing lactoferrin shRNA demonstrating reduced lactoferrin expression following induction of apoptosis in the knock-down cells is described in FIG. 3C. Moreover, FIG. 3D shows the results of a chemotaxis assay to determine neutrophil migration towards fMLP in the presence of supernatants obtained from control, lactoferrin shRNA and mock-transfected BL cells.

FIG. 4B shows neutrophil chemotaxis towards chemoattractants that were incubated with lactoferrin followed by the addition of neutralising anti-lactoferrin monoclonal antibody. Antibodies were subsequently removed using magnetic anti-IgG beads to demonstrate that lactoferrin's inhibitory effect was not due to modulation of chemoattractants.

8. Immunoblot analysis of lysates of neutrophils incubated with or without biotinylated lactoferrin at 37° C. for 1 h to demonstrate binding of lactoferrin to neutrophils is shown in Figure. Further, detailed analysis of direct binding of lactoferrin to neutrophils (Scatchard plot) is also demonstrated (see supplemental FIG. 2 from Bournazou, 2009).

A Chemotaxis assay to determine neutrophil migration in the presence of purified recombinant iron-depleted (apo), partially iron-saturated and fully iron-saturated (holo) recombinant lactoferrin is shown in FIG. 4H in paper.

FIG. 5C provides cytospin images of cells demonstrating inhibition of neutrophil migration by lactoferrin in vivo.

FIG. 6C provides results demonstrating the failure of lactoferrin to modulate changes in Intracellular Ca²⁺ levels in neutrophils responding to fMLP.

FIG. 7E shows that Lactoferrin appears to modulate ERK phosphorylation in neutrophils responding to fMLP.

The failure of primary necrotic cells to release lactoferrin is shown in FIG. 8F and the failure of necrotic cells to release mediators of neutrophil migration inhibition is shown in supplemental FIG. 3.

The Neutralisation of inhibition of neutrophil migration using monoclonal antibodies against lactoferrin is detailed in supplemental FIG. 1. All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A method of modulating granulocyte activation and/or migration towards a cell or population of cells, said method comprising modulating the amount of lactoferrin in the vicinity of said cells and/or said granulocytes.

2. The method according to claim 1, wherein said method is a method of inhibiting granulocyte migration towards said cell or population of cells.

3. The method according to claim 2, wherein the method comprises increasing the amount of lactoferrin in the vicinity of said cells and/or granulocytes.

4. The method according to claim 3, wherein said method comprises administration of lactoferrin or nucleic acid encoding lactoferrin to said cells.

5. The method according to claim 1, wherein activation of granulocytes is inhibited.

6. The method according to claim 5, wherein said granulocytes display reduction of cleavage of CD62L and reduction of expression of CD11b when compared to granulocytes in the absence of administration of lactoferrin or nucleic acid encoding lactoferrin.

7. The method according to claim 1, wherein polarisation of granulocytes is inhibited.

8-9. (canceled)

10. The method according to claim 2, wherein said population of cells comprises tumour cells.

11. (canceled)

12. The method according to claim 1, wherein said method is a method of enhancing granulocyte migration towards said cell or population of cells.

13. The method according to claim 12, wherein the method comprises administering an inhibitor of lactoferrin to said cells and/or granulocytes.

14. The method according to claim 12, wherein said population of cells comprises apoptotic cells.

15. The method according to claim 12, wherein said method induces or enhances an inflammatory response to said population of cells.

16. The method according to claim 12, wherein granulocyte mediated killing of said cells or population of cells is enhanced.

17. The method according to claim 12, wherein said population of cells comprises tumour cells.

18. The method according to claim 17, wherein the method is a method of treating cancer.

19. The method according to claim 1 wherein the granulocytes are neutrophils.

20-26. (canceled)

27. A pharmaceutical composition comprising a modulator of lactoferrin concentration or expression.

28. The pharmaceutical composition according to claim 27, wherein the composition comprises lactoferrin, a nucleic acid encoding lactoferrin, or an enhancer of lactoferrin activity or expression.

29. The pharmaceutical composition according to claim 28, wherein said composition is for the treatment of inflammatory disease.

30. The pharmaceutical composition according to claim 27, wherein the composition comprises a lactoferrin inhibitor.

31. The pharmaceutical composition according to claim 29, wherein said composition is for the treatment of cancer.

32. The pharmaceutical composition of claim 30, wherein the lactoferrin inhibitor is selected from the group consisting of an antibody and/or an antigen binding fragment thereof; and a sense or antisense nucleic acid sequence.

33. The method according to claim 13, wherein said population of cells comprises apoptotic cells.

34. The pharmaceutical composition according to claim 30, wherein said composition is for the treatment of cancer.

35. A method of treating inflammatory disease and/or cancer, said method comprising administering a modulator of lactoferrin concentration to a subject in need thereof.

36. The method according to claim 35, wherein the inflammatory disease is a chronic inflammatory disease.

37. The method of claim 36, wherein the chronic inflammatory disease is selected from the group consisting of vasculitis; pulmonary fibrosis and ischaemia reperfusion injury.

38. The method of claim 35, wherein the cancer is a cancer in which granulocytes, for example neutrophils, play a supportive role.

39. The method of claim 35, wherein the cancer is selected from the group consisting of gliomas; verroucous carcinoma; gastric carcinoma and melanoma.

40. The method of claim 35, wherein the modulator is a lactoferrin inhibitor.

41. The method of claim 35, wherein the modulator is a nucleic acid encoding lactoferrin, or an enhancer of lactoferrin activity or expression.

42. The method of claim 40, wherein the lactoferrin inhibitor is selected from the group consisting of an antibody and/or an antigen binding fragment thereof; and a sense or antisense nucleic acid sequence.
